(19) United States
(12) Patent Application Publication
(10) Pub. No.: US 2025/0297219 A1
(43) Pub. Date: Sep. 25, 2025

(54) METHODS FOR GENERATING ENGINEERED LYMPHOCYTES WITH ENRICHED T MEMORY STEM CELLS

(71) Applicant: Kite Pharma, Inc., Santa Monica, CA (US)

(72) Inventors: Beata Berent-Maoz, Los Angeles, CA (US); Yijun Liu, San Ramon, CA (US)

(21) Appl. No.: 19/045,894

(22) Filed: Feb. 5, 2025

Related U.S. Application Data
(60) Provisional application No. 63/551,459, filed on Feb. 8, 2024.

(51) Int. Cl. C12N 5/0783; A61K 35/17; A61K 40/11; A61K 40/31; A61K 40/32; A61K 40/42; C12N 5/00

(52) U.S. Cl. CPC C12N 5/0636; A61K 35/17; A61K 40/11; A61K 40/31; A61K 40/32; A61K 40/42; C12N 5/0087; C12N 5/562; C12N 2501/2302; C12N 2501/51; C12N 2501/515

(57) ABSTRACT

Provided herein are methods for manufacturing CAR-T cell products with high purity of TSCM subsets (>90%), independent of the variations from incoming leukapheresis. In some embodiments, to isolate the CCR7 and CD45RA double positive T cell subset, the processes described herein deplete CD45RO positive cells from leukapheresis and positively enrich for a CD4 and CD8 T cell population to isolation both TSCM and effector memory T cell (TEMRA) subsets, both of which positively express CD45RA and CCR7.

### CROSS-REFERENCE TO RELATED APPLICATIONS

This application claims priority to U.S. Provisional Application No. 63/551,459, filed Feb. 8, 2024, which is incorporated herein in its entirety for all purposes.

### TECHNICAL FIELD

The present disclosure relates to the field of cell therapy, and more specifically, compositions and methods for manufacturing engineered lymphocytes.

### BACKGROUND

Adoptive T cell therapy products with a high abundance of juvenile T cell population, in particular of the T memory stem cell (TSCM) subset, have gained increasing attention because of their characteristics of long-life span and ability to reconstitute the full spectrum of memory and effector T cell subsets. In addition, having a high percentage of juvenile T cell subset in an adoptive T cell therapy product has also been shown to correlate with a better clinical outcome.

However, the development of a process that robustly generates genetic engineered T cell with high purity of memory stem cell subset is still an unmet need in clinical manufacturing. Although it has been demonstrated that a shorter T cell expansion retains un-differentiated T phenotype, variations between donor to donor and/or patient to patient from the starting leukapheresis make the percentage of T cell memory stem cell subset in the final T cell product unpredictable.

In addition, clinical or commercial scale processing of leukapheresis involves bead-based process, which isn't compatible with complex T subset isolation typically associated with multiple steps of positive enrichment.

### SUMMARY

Described herein are processes that generate CAR-T cells with high purity of TSCM subset (>90%), independent of the variations from incoming leukapheresis material. This method leverages the understanding of T memory stem cell immunophenotypes, characterized by the positive expression of CD45RA and CCR7, and the negative expression of CD45RO. In some embodiments, to isolate the CCR7 and CD45RA double positive T cell subset, the processes described herein deplete CD45RO positive cells from leukapheresis and positively enrich for a CD4 and CD8 T cell population to isolation both TSCM and effector memory T cell (TEMRA) subsets, both of which positively express CD45RA and CCR7.

According to embodiments of the disclosure, TEMRA cells do not sustain a level of CD3 and CD28 activation and eventually die out during activation and transduction processes, thus leading to a final CAR-T cell population enriched with a high purity of TSCM subset (˜90%).

The methods described herein deliver a consistent and improved product profile associated with memory stem cell subset, e.g. % transduction efficiency, % juvenile T cells, and yield of T cells at harvest.

The methods herein represent a significant improvement over current CAR T-cell manufacture methods in their ability to generate CAR T-cell products with greatly increased percentages of TSCM cells. Such cells have stem-like capacities to expand and self-renew, while retaining plasticity. TSCM cells are also capable of reconstituting the entire spectrum of memory and effector T-cell subsets. In addition, the methods described herein result in CAR T-cell products with unprecedently high percentages of TSCM cells notwithstanding the fact that TSCM cells are a rare population in lymphocytes (2-3%).

An embodiment of the disclosure is related to a method for manufacturing transduced lymphocytes, including: depleting a population of cells expressing CD45RO from a sample of lymphocytes obtained from a donor subject; activating a population of lymphocytes expressing at least one of CD4 and CD8 from the sample of lymphocytes by stimulating the population of lymphocytes expressing at least one of CD4 and CD8 with at least one T cell stimulating agent; and incubating the population of lymphocytes expressing at least one of CD4 and CD8 with a polynucleotide vector to transduce the population of lymphocytes expressing at least one of CD4 and CD8 lymphocytes to produce transduced lymphocytes.

An embodiment of the disclosure is related to a population of cells prepared by any of the methods described herein, where at least 80% of the population of cells express CCR7 and CD45RA, and where at most 10% of the population of cells are a combination of effector memory T cells (TEM) and central memory T cells (TEM).

An embodiment of the disclosure is related to a pharmaceutical composition including the population of cells described above.

An embodiment of the disclosure is related to a method for administering T cells to a subject, including injecting to the subject a harvested sample prepared by the anyone of the methods described herein, or the pharmaceutical composition described above.

### BRIEF DESCRIPTION OF THE DRAWINGS

FIG. 1 is a diagram illustrating a process for generating CD45RO negative T cells from leukapheresis according to an embodiment of the disclosure.

FIG. 2 is schematic generally showing two competing processes for CAR T-cell manufacture according to an embodiment of the disclosure.

FIG. 3 is a series of plots showing flow cytometry results for T cell memory phenotype according to an embodiment of the disclosure.

FIG. 4 is a series of graphs showing flow cytometry results for a panel of memory markers of T cells according to an embodiment of the disclosure.

FIG. 5 shows bar graphs showing percentage of Tscm in leukapheresis and final CAR-T product from multiple donors according to an embodiment of the disclosure.

FIG. 6 shows a bar graph and a plot showing anti-CD19 and anti-CD20 bicistronic CAR expression according to an embodiment of the disclosure.

FIG. 7 shows bar graphs showing both total T cell yield (left) and CAR+ T cell yield (right) from multiple donors according to an embodiment of the disclosure.

FIG. 8 shows a series of graphs showing oxygen consumption rate from Seahorse T cell metabolic profiling assay according to an embodiment of the disclosure.

FIG. 9 shows bar graphs showing cytotoxicity of CAR T cells against Nalm6 target cells according to an embodiment of the disclosure.

FIG. 10 shows CAR T cell proliferation upon antigen stimulation from target cells line according to an embodiment of the disclosure.

FIG. 11 is a graph showing the percentage of naïve/memory stem cell subset of CD3 T cells from apheresis and CAR T cell production from a CD4,8 enrichment process versus a CD45RO deletion process according to an embodiment of the disclosure.

FIG. 12 is a graph showing the percentage of Treg cells in a CAR T-cell product according to an embodiment of the disclosure.

FIG. 13 shows a series of graphs showing the percentage killing of Nalm6-luc target cells at 1:1, 1:3, 1:9, and 1:27 effector to target ratio over 24 hours in coculture, according to an embodiment of the disclosure.

FIG. 14 shows a series of graphs showing cytokine concentration in supernatant from CAR T cells and Nalm6 target cells coculture assay at 1:1 effector to target ratio, according to an embodiment of the disclosure.

FIG. 15 shows a series of graphs showing the measurement of T cell respiration capacity and ATP product rate generated from glycolysis and mitochondrial pathways according to an embodiment of the disclosure.

FIG. 16 shows a series of graphs showing cytokine secretion index measured as the percentage of cells positive for IFN-gamma, TNF-alpha, and IL-2 multiplied by mean fluorescent intensity of those cytokines from flow cytometry assay from a repeat antigen stimulation assay according to an embodiment of the disclosure.

FIG. 17 is a graph showing the percentage positivity of exhaustion markers, including CD39, LAG3, PD1, TIGIT, TIM3, and memory markers, including CD27 and CD62L, of CAR T cell products that are repeatedly stimulated by Nalm6 target cells for 5 times over 2.5 weeks, according to an embodiment of the disclosure.

### DETAILED DESCRIPTION

### Definitions

In order for the present disclosure to be more readily understood, certain terms are first defined below. Additional definitions for the following terms and other terms are set forth throughout the Specification.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless specifically stated or evident from context the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

“Administering” refers to the physical introduction of an agent to a subject, such as a modified T cell disclosed herein, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “allogeneic” refers to any material derived from one individual which is then introduced to another individual of the same species, e.g., allogeneic T cell transplantation.

The term “autologous” refers to a therapeutic intervention that uses an individual's own cells or tissues, which are processed outside the body, and reintroduced into the individual.

The term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

An “antigen binding molecule,” “antigen binding portion,” “antigen binding fragment,” or “antibody fragment” refers to any molecule that comprises the antigen binding parts (e.g., CDRs) of the antibody from which the molecule is derived. An antigen binding molecule can include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e.,

Fc fusion molecules comprising peptide binding domains) are another example of suitable antigen binding molecule. In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments an antigen binding molecule is a chimeric antigen receptor (CAR) or an engineered T cell receptor (TCR).

The term “variable region” or “variable domain” is used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

“Chimeric antigen receptor” or “CAR” refers to a molecule engineered to comprise a binding motif and a means of activating immune cells (for example T cells such as naive T cells, central memory T cells, effector memory T cells or combination thereof) upon antigen binding. CARs are also known as artificial T cell receptors, chimeric T cell receptors or chimeric immunoreceptors. In some embodiments, a CAR comprises a binding motif, an extracellular domain, a transmembrane domain, one or more co-stimulatory domains, and an intracellular signaling domain. A T cell that has been genetically engineered to express a chimeric antigen receptor may be referred to as a CAR T cell. “Extracellular domain” (or “ECD”) refers to a portion of a polypeptide that, when the polypeptide is present in a cell membrane, is understood to reside outside of the cell membrane, in the extracellular space.

A “T cell receptor” or “TCR” refers to antigen-recognition molecules present on the surface of T cells. During normal T cell development, each of the four TCR genes, α, β, γ, and δ, may rearrange leading to highly diverse TCR proteins.

The term “heterologous” means from any source other than naturally occurring sequences. For example, a heterologous sequence included as a part of a costimulatory protein is amino acids that do not naturally occur as, i.e., do not align with, the wild type human costimulatory protein. For example, a heterologous nucleotide sequence refers to a nucleotide sequence other than that of the wild type human costimulatory protein-encoding sequence.

Term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-100%).

The immune cells of the immunotherapy can come from any source known in the art. For example, immune cells can be differentiated in vitro from a hematopoietic stem cell population, or immune cells can be obtained from a subject. Immune cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the immune cells can be derived from one or more immune cell lines available in the art. Immune cells can also be obtained from a unit of blood collected from a subject using any number of techniques known to the skilled artisan, such as FICOLL™ separation, OPTIPREP™ separation, and/or apheresis. Additional methods of isolating immune cells for an immune cell therapy are disclosed in U.S. Patent Publication No. 2013/0287748, which is herein incorporated by reference in its entirety.

A “patient” includes any human who is afflicted with a cancer (e.g., a lymphoma or a leukemia). The terms “subject” and “patient” are used interchangeably herein.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The terms “reducing” and “decreasing” are used interchangeably herein and indicate any change that is less than the original. “Reducing” and “decreasing” are relative terms, requiring a comparison between pre-and post-measurements. “Reducing” and “decreasing” include complete depletions.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “transduction” and “transduced” refer to the process whereby foreign nucleic acid is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

The term “7-day process” refers to a CAR cell manufacturing process which takes about 7 days following initial enrichment and/or activation step(s). The 7-day process is at least 8 days in length from the initial enrichment and/or activation step(s) to a harvesting step, and can be between 8 to 11 days in total when including the enrichment and/or activation step(s).

The term “5-day process” refers to a CAR cell manufacturing process which takes about 5 days following initial enrichment and/or activation step(s). The 5-day process is 6 days in length from the initial enrichment and/or activation step(s) to a harvesting step, and can be between 6 to 9 days in total when including the enrichment and/or activation step(s).

The term “3-day process” refers to a CAR cell manufacturing process which takes up to 3 days from initial enrichment and/or activation step(s). The 3-day process is about 4 days in length from the initial enrichment and/or activation step(s) to a harvesting step. The 3-day process does not include a cell expansion step comprising one or more days following a transduction step and preceding a harvesting step.

In some embodiments, the 3-day process described herein is about 5 days in length from the initial enrichment and/or activation step(s) to a harvesting step. In some embodiments, the 3-day process is about 3 to 4 days in length or about 72 to 96 hours in length from the initial enrichment and/or activation step(s) to a harvesting step (e.g., about 72 hours, 74 hours, 76 hours, 78 hours, 80 hours, 82 hours, 84 hours, 86 hours, 88 hours, 90 hours, 92 hours, 94 hours, 96 hours in length). In some embodiments, the 3-day process is about 1 to 2 days in length or about 24 to 48 hours in length from the initial enrichment and/or activation step(s) to a harvesting step (e.g., about 24 hours, 26 hours, 28 hours, 30 hours, 32 hours, 34 hours, 36 hours, 38 hours, 40 hours, 42 hours, 44 hours, 46 hours, 48 hours in length). In some embodiments, the 3-day process is about 2 to 3 days in length or about 48 to 72 hours in length from the initial enrichment and/or activation step(s) to a harvesting step (e.g., about 48 hours, 50 hours, 52 hours, 54 hours, 56 hours, 58 hours, 60 hours, 62 hours, 64 hours, 66 hours, 68 hours, 70 hours, 72 hours in length). In some embodiments, the 3-day process is about 4 to 5 days in length or about 96 to about 120 hours in length from the initial enrichment and/or activation step(s) to a harvesting step (e.g., about 96 hours, 98 hours, 100 hours, 102 hours, 104 hours, 106 hours, 108 hours, 110 hours, 112 hours, 114 hours, 116 hours, 118 hours, 120 hours in length). In some embodiments, the 3-day process is less than 5 days or 120 hours in length from the initial enrichment and/or activation step(s) to a harvesting step.

### Preparation of Engineered Lymphocytes

The conventional autologous CAR cell manufacturing process takes about 7 days and can be much longer. The lengthy process was believed to be required at least because of the limited supply of starting materials, i.e., lymphocytes obtained from an apheresis collection from a donor subject, the relatively low-efficiency transduction, and the need to expand the transduced cells. Non-limiting examples of CAR cell manufacturing processes are described in patent publications WO2015120096, WO2016191755, and WO2023230276 each of which is incorporated herein in its entirety.

The instant disclosure describes improvements to the conventional CAR T-cell manufacturing process. Specifically, the instant disclosure is related to methods for increasing the percentage of memory stem cells in a final CAR T-cell population regardless of the characteristics of a starting leukapheresis sample, thus resulting in a final product with increased efficacy over traditional CAR T-cell products.

An embodiment of the disclosure is related to a method for manufacturing transduced lymphocytes, including: depleting a population of cells expressing CD45RO from a sample of lymphocytes obtained from a donor subject; activating a population of lymphocytes expressing at least one of CD4 and CD8 from the sample of lymphocytes by stimulating the population of lymphocytes expressing at least one of CD4 and CD8 with at least one T cell stimulating agent; and incubating the population of lymphocytes expressing at least one of CD4 and CD8 with a polynucleotide vector to transduce the population of lymphocytes expressing at least one of CD4 and CD8 lymphocytes to produce transduced lymphocytes.

An embodiment of the disclosure is related to the method above, where depleting the population of cells expressing CD45RO from the sample of lymphocytes includes: contacting the sample of lymphocytes with an anti-hCD45RO antibody and bead conjugate so as to generate a labeled population of cells expressing CD45RO; and separating the labeled population of cells expressing CD45RO from the sample of lymphocytes. Alternatively, in some embodiments, depleting the population of cells expressing CD45RO from the sample of lymphocytes includes contacting the sample of lymphocytes, leukapheresis, or PBMC with an anti-hCD45RO biotin or anti-hCD45RO PE/FITC/APC followed by further contacting the sample with anti-biotin microbead or anti-PE/FITC/APC to generate a magnetically labeled population of cells expressing CD45RO. Alternatively, in some embodiments, depleting the population of cells expressing CD45RO from the sample of lymphocytes includes labeling the sample with anti-hCD45 antibody with a fluorescent fluorophore followed by flow cytometry or microfluidics device-based sorting. Alternatively, in some embodiments, depleting the population of cells expressing CD45RO from the sample of lymphocytes includes contacting the sample of lymphocytes with an anti-hCD45RA antibody and bead conjugate so as to generate a labeled population of cells expressing CD45RA, and separating the labeled population of cells expressing CD45RA from the sample of lymphocytes.

An embodiment of the disclosure is related to any of the methods above, further including enriching the sample of lymphocytes for a population of lymphocytes expressing at least one of CD4 and CD8. In such an embodiment, enriching the sample of lymphocytes for the population of lymphocytes expressing at least one of CD4 and CD8 includes: contacting the sample of lymphocytes with at least one of an anti-CD4 antibody and bead conjugate, and an anti-CD8 antibody and bead conjugate so as to generate a labeled population of cells expressing at least one of CD4 and CD8; and isolating the labeled population of cells expressing at least one of CD4 and CD8 from the sample of lymphocytes.

An embodiment of the disclosure is related to any of the methods above, where the step of activating the population of lymphocytes expressing at least one of CD4 and CD8 by stimulating the population of lymphocytes expressing at least one of CD4 and CD8 with at least one T cell stimulating agent is done prior to incubating the population of lymphocytes expressing at least one of CD4 and CD8 with the polynucleotide vector. In some such embodiments, the at least one T cell stimulating agent includes an anti-CD3 antibody, an anti-CD28 antibody, or Interleukin-2. In some such embodiments, the activating is carried out in a closed system.

An embodiment of the disclosure is related to any of the methods above, where the population of lymphocytes expressing at least one of CD4 and CD8 are incubated with the at least one T cell stimulating agent for up to 72 hours.

An embodiment of the disclosure is related to any of the methods above, where the incubating is carried out in a closed system.

An embodiment of the disclosure is related to any of the methods above, where at least 80% of the transduced lymphocytes express CCR7 and CD45RA.

An embodiment of the disclosure is related to any of the methods above, where at least 90% of the transduced lymphocytes express CCR7 and CD45RA.

An embodiment of the disclosure is related to any of the methods above, where at most 10% of the transduced lymphocytes are a combination of effector memory T cells (TEM) and central memory T cells (TEM).

An embodiment of the disclosure is related to any of the methods above, where at most 5% of the transduced lymphocytes are a combination of effector memory T cells (TEM) and central memory T cells (TEM).

An embodiment of the disclosure is related to any of the methods above, further including: culturing the sample including the transduced lymphocytes for up to 72 hours before the lymphocytes are harvested to produce a harvested sample.

An embodiment of the disclosure is related to any of the methods above, where the transduced lymphocytes are cultured for less than 48 hours before being harvested.

An embodiment of the disclosure is related to any of the methods above, where the transduced lymphocytes are cultured for less than 36 hours before being harvested.

An embodiment of the disclosure is related to any of the methods above, where the culturing is carried out in a closed system.

An embodiment of the disclosure is related to any of the methods above, where the closed system has an inner surface area of at least 170 cm².

An embodiment of the disclosure is related to any of the methods above, where the closed system has an inner surface coated with a recombinant human fibronectin, where the coating is carried out with a solution that includes about 1-10 μg/ml of the recombinant human fibronectin.

An embodiment of the disclosure is related to any of the methods above, where the sample in the closed system includes at least 1.2×10⁸ lymphocytes.

An embodiment of the disclosure is related to any of the methods above, where the sample includes at least 0.6×10⁸ lymphocytes.

An embodiment of the disclosure is related to any of the methods above, further including, following the harvesting, administering the harvested lymphocytes to a subject in need thereof or freezing the harvested lymphocytes.

An embodiment of the disclosure is related to any of the methods above, where a total of 5,000 to 1,000,000 harvested lymphocytes per kilogram of the subject in need thereof are administered to the subject.

An embodiment of the disclosure is related to any of the methods above, where the sample of lymphocytes derived from the donor subject are washed leukapheresis cells, peripheral blood mononuclear cells (PBMCs) or T cells.

An embodiment of the disclosure is related to any of the methods above, where the polynucleotide vector is a viral vector.

An embodiment of the disclosure is related to any of the methods above, where the viral vector is a retroviral vector or a lentiviral vector.

An embodiment of the disclosure is related to any of the methods above, where the polynucleotide vector is a transposon.

An embodiment of the disclosure is related to any of the methods above, where the polynucleotide vector encodes a chimeric antigen receptor (CAR) or a T cell receptor (TCR).

An embodiment of the disclosure is related to any of the methods above, where the CAR or the TCR recognizes a tumor antigen. In such an embodiment, the tumor antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRVIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, Fc receptor-like protein 5, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RUI, RU2 (AS), surface adhesion molecule, survivin and telomerase, TAG-72, TACI, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), GPC3 (Glypican 3), EphA2, GPC2, CD133, LGR5, TROP2, CD146, CLDN3, CLDN4, CLDN6, CLDN9, CLDN18.2, CD70, TnMUC1, Alkaline phosphatase (placental type), MUC16, MUC17, MARTI, Pmel17, Melanoma-associated chondroitin sulfate proteoglycan, as well as any derivate or variant of these antigens.

An embodiment of the disclosure is related to a population of cells prepared by anyone of the methods described above, where at least 80% of the population of cells express CCR7 and CD45RA, and where at most 10% of the population of cells are a combination of effector memory T cells (TEM) and central memory T cells (TEM).

An embodiment of the disclosure is related to a pharmaceutical composition including the population of cells described above.

An embodiment of the disclosure is related to a method for administering T cells to a subject, including injecting to the subject a harvested sample prepared by the anyone of the methods described above, or the pharmaceutical composition described above.

An embodiment of the disclosure is related to the method for administering T cells to a subject described above, where the subject has cancer.

An embodiment of the disclosure is related to the method for administering T cells to a subject described above, where the cancer is a lung cancer, a GI cancer, a breast cancer, a gynecologic malignancy, a genitourinary malignancy, a neurologic tumor, a melanoma, a sarcoma, a pediatric cancer, an endocrine malignancy, Kaposi sarcoma, a Non-Hodgkin's Lymphoma, or mesothelioma.

An embodiment of the disclosure is related to the method for administering T cells to a subject described above, where the cancer is a B cell malignancy.

An embodiment of the disclosure is related to the method for administering T cells to a subject described above, where the cancer is Non-Hodgkin's Lymphomas (NHL), Diffuse Large B Cell Lymphoma (DLBCL), Small lymphocytic lymphoma (SLL/CLL), Mantle cell lymphoma (MCL), Follicular lymphoma (FL), Marginal zone lymphoma (MZL), Extranodal (MALT lymphoma), Nodal (Monocytoid B-cell lymphoma), Splenic, Diffuse large cell lymphoma, B cell chronic lymphocytic leukemia/lymphoma, Burkitt's lymphoma, Lymphoblastic lymphoma, acute myeloid leukemia, or multiple myeloma.

Embodiments of the disclosure are related to improved processes that can be completed within 5, 3, or even 1 day, following an enrichment step. In various examples, the 5-day process includes transduction preparation and implementation steps with a higher number of lymphocytes in contact with vectors immobilized to recombinant fibronectin coated to the inner surface of a closed system. Such an improved transduction procedure allows a much-abbreviated post-transduction cell expansion step.

Yet a further improved process, which does not require post-transduction expansion at all, can be completed within only 1, 2, up to 3 days, following an enrichment step.

In accordance with one embodiment of the present disclosure, therefore, provided is a method for preparing transduced (or transfected, in likewise manner) lymphocytes. In some embodiments, the method entails incubating a sample of lymphocytes with a polynucleotide vector to transduce the lymphocytes to produce transduced lymphocytes and culturing the sample that contains the transduced lymphocytes before the lymphocytes are harvested to produce a harvested sample.

In some embodiments, the culturing step is shortened as compared to the conventional process which takes about 4 days. In some embodiment, the culturing step is completed within 96 hours, or within 72 hours, 60 hours, 50 hours, 48 hours, 42 hours, 36 hours, 30 hours, 29 hours, 28 hours, 27 hours, 26 hours, 25 hours, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, or 4 hours.

In some embodiments, the time for the culturing step is counted from completion of the transduction step (e.g., removal of the cells from the system with immobilized vectors) to harvesting of the cells for storage, transport, or clinical use.

Culturing of transduced lymphocytes can be done in media and conditions known in the art. In some embodiments, the culturing of the transduced lymphocytes may be performed at a temperature and/or in the presence of CO₂. In certain embodiments, the temperature may be about 34° C., about 35° C., about 36° C., about 37° C., about 38° C., or about 39° C. In certain embodiments, the temperature may be about 34-39° C. In certain embodiments, the predetermined temperature may be from about 35-37° C. In certain embodiments, the preferred predetermined temperature may be from about 36-38° C. In certain embodiments, the predetermined temperature may be about 36-37° C. or more preferably about 37° C.

In some embodiments, culturing of the transduced lymphocytes may be performed in the presence of a predetermined level of CO₂. In certain embodiments, the predetermined level of CO₂ may be 1.0-10% CO₂. In certain embodiments, the predetermined level of CO₂ may be about 1.0%, about 2.0%, about 3.0%, about 4.0%, about 5.0%, about 6.0%, about 7.0%, about 8.0%, about 9.0%, or about 10.0% CO₂. In certain embodiments, the predetermined level of CO₂ may be about 4.5-5.5% CO₂. In certain embodiments, the predetermined level of CO₂ may be about 5% CO₂. In certain embodiments, the predetermined level of CO₂ may be about 3.5%, about 4.0%, about 4.5%, about 5.0%, about 5.5%, or about 6.5% CO₂. In some embodiments, the step of expanding the population of transduced T cells may be performed at a predetermined temperature and/or in the presence of a predetermined level of CO₂ in any combination. For example, in one embodiment, the step of expanding the population of transduced T cells may comprise a predetermined temperature of about 36-38° C. and in the presence of a predetermined level of CO₂ of about 4.5-5.5% CO₂.

Any suitable culture medium T cell growth media may be used for culturing the cells in suspension. For example, a T cell growth media may include, but is not limited to, a sterile, low glucose solution that includes a suitable amount of buffer, magnesium, calcium, sodium pyruvate, and sodium bicarbonate. In one embodiment, the culturing media is OpTmizer™ (Life Technologies), but one skilled in the art would understand how to generate similar media. In one embodiment, the culturing media is EX-VIVO™ serum free media (Lonza Bioscience).

The incubation (and/or transduction) step can be carried out in a closed system, without limitation. In certain embodiments, the closed system is a closed bag culture system, using any suitable cell culture bags (e.g., Mitenyi Biotec MACS® GMP Cell Differentiation Bags, Origen Biomedical PermaLife™ Cell Culture bags). In some embodiments, the closed system has an inner surface area of at least 500 cm². In some embodiments, the closed system has an inner surface area of at least 1000 cm², 1200 cm², 1400 cm², 1500 cm², 1600 cm², 1800 cm², 2000 cm², 2200 cm², 2500 cm², or 3000 cm². In some embodiments, the closed system has an inner surface area of not greater than 1500 cm², 1600 cm², 1800 cm², 2000 cm², 2200 cm², 2500 cm², or 3000 cm². In certain embodiments, the closed system is a closed bioreactor system (e.g., Xuri Cell Expansion System W25, CliniMACS®Prodigy, Cocoon® Platform).

In some embodiments, the cell culture bags used in the closed system are coated with a recombinant human fibronectin protein. The recombinant human fibronectin fragment may include three functional domains: a central cell-binding domain, heparin-binding domain II, and a CS1-sequence. The recombinant human fibronectin protein or fragment thereof may be used to increase gene efficiency of viral transduction of immune cells by aiding co-localization of target cells or the vector. In certain embodiments, the recombinant human fibronectin fragment is RetroNectin® (Takara Bio, Japan). In certain embodiments, the cell culture bags may be coated with recombinant human fibronectin fragment at a concentration of about 0.1-60 μg/mL, preferably 0.5-40 μg/mL. In certain embodiments, the cell culture bags may be coated with recombinant human fibronectin fragment at a concentration of about 0.5-20 μg/mL, 20-40 μg/mL, or 40-60 μg/mL. In certain embodiments, the cell culture bags may be coated with about 0.5 μg/mL, 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, or about 20 μg/mL recombinant human fibronectin fragment. In certain embodiments, the cell culture bags may be coated with about 2-5 μg/mL, about 2-10 μg/mL, about 2-20 μg/mL, about 2-25 μg/mL, about 2-30 μg/mL, about 2-35 μg/mL, about 2-40 μg/mL, about 2-50 μg/mL, or about 2-60 μg/mL recombinant human fibronectin fragment. In certain embodiments, the cell culture bags may be coated with at least about 2 μg/mL, at least about 5 μg/mL, at least about 10 μg/mL, at least about 15 μg/mL, at least about 20 μg/mL, at least about 25 μg/mL, at least about 30 μg/mL, at least about 40 μg/mL, at least about 50 μg/mL, or at least about 60 μg/mL recombinant human fibronectin fragment. In certain embodiments, the cell culture bags may be coated with at least about 10 μg/mL recombinant human fibronectin fragment. In certain embodiments, the cell culture bags may not be coated with recombinant human fibronectin fragment.

In some embodiments, a transduction enhancing agent is introduced into the closed system. Non-limiting examples of such transduction enhancing agents include Vectofusin™ transduction mixtures.

In certain embodiments, the cell culture bags used in the closed bag culture system may be blocked with human albumin serum (HSA). In an alternative embodiment, the cell culture bags are not blocked with HSA.

Once the closed system is coated with the recombinant fibronectin, a solution that includes the vector is added to the closed system so that the vector can be immobilized by the recombinant fibronectin, on the inner surface of the closed system. Such immobilization can improve the transduction efficiency once the cells are added.

In some embodiments, the vectors can be viral vectors, such as lentiviral vectors, as well as retroviral vectors. Several recombinant viruses have been used as viral vectors to deliver genetic material to a cell. Viral vectors that may be used in accordance with the transduction step may be any ecotropic or amphotropic viral vector including, but not limited to, recombinant retroviral vectors, recombinant lentiviral vectors, recombinant adenoviral vectors, and recombinant adeno-associated viral (AAV) vectors. In one embodiment, the viral vector is an MSGVI gamma retroviral vector. In some embodiments, the vectors are non-viral vectors.

In some embodiments, a total volume of at least 100 mL of the solution that contains the vector is used. In some embodiments, a total volume of at least 110 mL, 120 mL, 130 mL, 140 mL, 150 mL, 160 mL, 170 mL, 180 mL, 190 mL, 200 mL, 210 mL, 220 mL, 230 mL, 240 mL, 250 mL, 260 mL, 270 mL, 280 mL, 290 mL, 300 mL, 350 mL, or 400 mL of the solution that contains the vector is used. In some embodiments, a total volume of no more than 150 mL, 160 mL, 170 mL, 180 mL, 190 mL, 200 mL, 210 mL, 220 mL, 230 mL, 240 mL, 250 mL, 260 mL, 270 mL, 280 mL, 290 mL, 300 mL, 350 mL, 400 mL, or 500 mL of the solution that contains the vector is used.

In some embodiments, the vector solution includes at between 1×10³ to 1×10¹² transduction units per milliliter (TU/ml) of the viral vector.

Once the closed system is coated with the recombinant fibronectin and has immobilized the vector, the vector solution can be removed. In some embodiments, the closed system does not include recombinant fibronectin. In some embodiments, the removal of the vector solution is done by gravity or syringe drain, which helps to retain the immobilized vector on the inner surface while removing impurities. Lymphocyte transduction can be carried in the coated closed system with the immobilized vectors. In some embodiments, the transduction is performed with a sample that contained the lymphocytes. In some embodiments, the sample includes at least 2.5×10⁷ lymphocytes (e.g., T cells). In some embodiments, the sample includes at least 3×10⁷, 4×10⁷, 5×10⁷, 6×10⁷, 7×10⁷, 8×10⁷, 9×10⁷, 1×10⁸, 1.2×10⁸, 1.5×10⁸, 1.8×10⁸, 2×10⁸, 2.2×10⁸, 2.5×10⁸, 2.6×10⁸, 2.7×10⁸, 2.8×10⁸, 2.9×10⁸, 3×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.5×10⁸, 8×10⁸, 9×10⁸, or 10×10⁸ lymphocytes (e.g., T cells). In some embodiments, the sample includes no more than 3×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.5×10⁸, 8×10⁸, 9×10⁸, or 10×10⁸ lymphocytes (e.g., T cells).

In some embodiments, lymphocyte transduction is carried out in a closed system that does not contain an immobilizing agent (e.g., recombinant fibronectin).

The lymphocytes used in the presently disclosed methods are typically obtained from a donor subject, which may be a cancer patient that is to be treated with a population of cells generated by the methods described herein (i.e., an autologous donor), or may be an individual that donates a lymphocyte sample that, upon generation of the population of cells generated by the methods described herein, will be used to treat a different individual or cancer patient (i.e., an allogeneic donor). The lymphocytes may be obtained from the donor subject by any suitable method used in the art. For example, the lymphocytes may be obtained by any suitable extracorporeal method, venipuncture, or other blood collection method by which a sample of blood and/or lymphocytes is obtained. In one embodiment, the lymphocytes are obtained by apheresis.

Optionally, in some embodiments, the method described herein further includes a step of enriching a population of lymphocytes obtained from the donor subject, prior to the transduction. The donor subject may be a cancer patient that is to be treated with a population of cells generated by the methods described herein (i.e., an autologous donor), or may be an individual that donates a lymphocyte sample that, upon generation of the population of cells generated by the methods described herein, will be used to treat a different individual or cancer patient (i.e., an allogeneic donor). The population of lymphocytes may be obtained from the donor subject by any suitable method used in the art. For example, the population of lymphocytes may be obtained by any suitable extracorporeal method, venipuncture, or other blood collection method by which a sample of blood and/or lymphocytes is obtained. In one embodiment, the population of lymphocytes is obtained by apheresis. Enrichment of lymphocytes may be accomplished by any suitable separation method including, but not limited to, the use of a separation medium (e.g., Ficoll-Paque™, RosetteSep™ HLA Total Lymphocyte enrichment cocktail, Lymphocyte Separation Medium (LSA) (MP Biomedical Cat. No. 0850494X), a non-ionic iodixanol-based medium such as OptiPrep™, or the like), cell size, shape or density separation by filtration or elutriation, immunomagnetic separation (e.g., magnetic-activated cell sorting system, MACS), fluorescent separation (e.g., fluorescence activated cell sorting system, FACS), or bead-based column separation.

In some embodiments, circulating lymphoma cells are removed from the sample through positive enrichment for CD⁴⁺/CD⁸⁺ cells via the use of selection reagents. In some such embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

In some such embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity-or immunoaffinity-based separation. For example, the isolation in some embodiments includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps may be based on positive selection, in which the cells having bound the reagents are retained for further use, and/or negative selection, in which the cells having not bound to the antibody or binding partner are retained. In some examples, both fractions are retained for further use.

In some such embodiments, negative selection may be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step may deplete cells expressing multiple markers simultaneously, such as by incubating cells with a plurality of antibodies or binding partners, each specific for a marker targeted for negative selection. Likewise, multiple cell types may simultaneously be positively selected by incubating cells with a plurality of antibodies or binding partners expressed on the various cell types.

For example, in some embodiments, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+T cells, are isolated by positive or negative selection techniques. For example, CD3+, CD28+T cells may be positively selected using anti-CD3/anti-CD28 conjugated magnetic beads (e.g., DYNABEADS® M-450 CD3/CD28 T Cell Expander). In some embodiments, the population of cells is enriched for T cells with naïve phenotype (CD45RA+CCR7+).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (markerhigh) on the positively or negatively selected cells, respectively.

In particular embodiments, a biological sample, e.g., a sample of PBMCs or other white blood cells, are subjected to selection of CD4+ T cells, where both the negative and positive fractions are retained. In certain embodiments, CD8+ T cells are selected from the negative fraction. In some embodiments, a biological sample is subjected to selection of CD8+ T cells, where both the negative and positive fractions are retained. In certain embodiments, CD4+ T cells are selected from the negative fraction.

In some embodiments, T cells are separated from a PBMC sample by negative selection of markers expressed on non-T cells, such as B cells, monocytes, or other white blood cells, such as CD14. In some embodiments, a CD4+or CD8+selection step is used to separate CD4+helper and CD8+cytotoxic T cells. Such CD4+and CD8+populations may be further sorted into sub-populations by positive or negative selection for markers expressed or expressed to a relatively higher degree on one or more naive, memory, and/or effector T cell subpopulations.

In one example, to enrich for CD4+cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques. In some embodiments, the sample or composition of cells to be separated is incubated with small, magnetizable or magnetically responsive material, such as magnetically responsive particles or microparticles, such as paramagnetic beads (e.g., such as Dynabeads™ or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample. In some embodiments, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some embodiments, a combination of positive and negative selection is performed during the same selection step, where the positive and negative fractions are retained and further processed or subject to further separation steps. In some embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody-or other binding partner (e.g., streptavidin)-coated magnetic particles, are added. In certain embodiments, streptavidin-coated magnetic particles are used in conjunction with biotinylated primary or secondary antibodies. In some embodiments, the magnetically responsive particles are left attached to the cells that are to be subsequently incubated, cultured and/or engineered; in some embodiments, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they may be eluted and recovered. In certain embodiments, the non-target cells are labelled and depleted from the heterogeneous population of cells.

In some embodiments, the isolation or separation is carried out using a system, device, or apparatus that carries out one or more of the isolation, cell preparation, separation, processing, incubation, culture, and/or formulation steps of the methods. In some embodiments, the system is used to carry out each of these steps in a closed or sterile environment, for example, to minimize error, user handling and/or contamination. In one example, the system is a system as described in International Patent Application, Publication Number WO2009/072003, or U.S. Pat. No. 20,110,003380 A1, which are each incorporated herein by reference. In some embodiments, the system or apparatus carries out one or more, e.g., of the isolation, processing, engineering, and formulation steps in an integrated or self-contained system, and/or in an automated or programmable fashion. In some embodiments, the system or apparatus includes a computer and/or computer program in communication with the system or apparatus, which allows a user to program, control, assess the outcome of, and/or adjust various embodiments of the processing, isolation, engineering, and formulation steps. In some embodiments, the separation and/or other steps is carried out using CliniMACS® system (Miltenyi Biotec), for example, for automated separation of cells on a clinical-scale level in a closed and sterile system. Components may include an integrated microcomputer, magnetic separation unit, peristaltic pump, and various pinch valves. The integrated computer in some embodiments controls components of the instrument and directs the system to perform repeated procedures in a standardized sequence. The magnetic separation unit in some embodiments includes a movable permanent magnet and a holder for the selection column. The peristaltic pump controls the flow rate throughout the tubing set and, together with the pinch valves, ensures the controlled flow of buffer through the system and continual suspension of cells.

The CliniMACS® system in some embodiments uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS® Prodigy system (Miltenyi Biotec). The CliniMACS® Prodigy system in some embodiments is equipped with a cell processing unity that permits automated washing and fractionation of cells by centrifugation. The CliniMACS® Prodigy system may also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS® Prodigy system may also include an integrated cell cultivation chamber which accomplishes cell culture protocols such as, e.g., cell differentiation and expansion, antigen loading, and long-term cell culture. Input ports may allow for the sterile removal and replenishment of media and cells may be monitored using an integrated microscope.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5): 355-376. In both cases, cells may be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, at least 0.5×10⁹ lymphocytes are acquired from the donor, and are optionally enriched and/or subjected to the stimulation. In some embodiments, at least 0.6×10⁹, 0.7×10⁹, 0.8×10⁹, 0.9×10⁹, 1×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2×10⁹, 2.5×10⁹, or 3×10⁹ lymphocytes are acquired from the donor, and are optionally enriched and/or subjected to the stimulation. In some embodiments, no more than 1×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2×10⁹, 2.5×10⁹, or 3×10⁹ lymphocytes are acquired from the donor, and are optionally enriched and/or subjected to the stimulation.

The methods described herein further includes a step of stimulating the lymphocytes with one or more lymphocyte stimulating agents. In some embodiments, the stimulation is performed prior to the transduction step. In some embodiments, the stimulation is performed after the transduction step. The stimulation step is also referred to herein as an activation step.

Any combination of one or more suitable lymphocyte stimulating agents may be used to stimulate (activate) the lymphocytes. Non-limiting examples include an antibody or functional fragment thereof which targets a T-cell stimulatory or co-stimulatory molecule (e.g., anti-CD2 antibody, anti-CD3 antibody, anti-CD28 antibody, or functional fragments thereof) a T cell cytokine (e.g., any isolated, wildtype, or recombinant cytokines such as: interleukin 1 (IL-1), interleukin 2, (IL-2), interleukin 4 (IL-4), interleukin 5 (IL-5), interleukin 7 (IL-7), interleukin 15 (IL-15), interleukin 21 (IL-21), transforming growth factor-β (TGF-β), tumor necrosis factor a (TNFα)), or any other suitable mitogen (e.g., tetradecanoyl phorbol acetate (TPA), phytohaemagglutinin (PHA), concanavalin A (conA), lipopolysaccharide (LPS), pokeweed mitogen (PWM)) or natural ligand to a T-cell stimulatory or co-stimulatory molecule. In some embodiments, the stimulating agent is an anti-CD3 antibody and/or an anti-CD28 antibody.

In some embodiments, the lymphocyte stimulating agent may be a bead-based activator, such as T-cell TransAct™ (Miltenyi Biotec), Dynabeads® (Thermo Fisher Scientific), or Cloudz™ T Cell Activation Kit (R&D Systems).

In some embodiments, the step of stimulating lymphocytes as described herein may entail stimulating the lymphocytes with one or more stimulating agents at a predetermined temperature, for a predetermined amount of time, and/or in the presence of a predetermined level of CO₂. In certain embodiments, the predetermined temperature for stimulation may be about 34° C., about 35° C., about 36° C., about 37° C., about 38° C., or about 39° C. In certain embodiments, the predetermined temperature for stimulation may be about 34-39° C. In certain embodiments, the step of stimulating the lymphocytes comprises stimulating the lymphocytes with one or more stimulating agents for a predetermined time. In certain embodiments, the predetermined time for stimulation may be about 24-72 hours. In certain embodiments, the predetermined time for stimulation may be about 24-36 hours. In certain embodiments, the step of stimulating the lymphocytes may comprise stimulating the lymphocytes with one or more stimulating agents in the presence of a predetermined level of CO₂. In certain embodiments, the predetermined level of CO₂ for stimulation may be about 1.0-10% CO₂. In certain embodiments, the predetermined level of CO₂ for stimulation may be about 1.0%, about 2.0%, about 3.0%, about 4.0%, about 5.0%, about 6.0%, about 7.0%, about 8.0%, about 9.0%, or about 10.0% CO₂.

In some embodiments, an anti-CD3 antibody (or functional fragment thereof), an anti-CD28 antibody (or functional fragment thereof), or a combination of anti-CD3 and anti-CD28 antibodies may be used in accordance with the step of stimulating the population of lymphocytes. Any soluble or immobilized anti-CD3 and/or anti-CD28 antibody or functional fragment thereof may be used (e.g., clone OKT3 (anti-CD3), clone 145-2C11 (anti-CD3), clone UCHT1 (anti-CD3), clone L293 (anti-CD28), clone 15E8 (anti-CD28)). In some aspects, the antibodies may be purchased commercially from vendors known in the art including, but not limited to, Miltenyi Biotec, BD Biosciences (e.g., MACS GMP CD3 pure 1 mg/mL, Part No. 170-076-116), and eBioscience, Inc. Further, one skilled in the art would understand how to produce an anti-CD3 and/or anti-CD28 antibody by standard methods. Any antibody used in the methods described herein should be produced under Good Manufacturing Practices (GMP) to conform to relevant agency guidelines for biologic products.

In certain embodiments, the T cell stimulating agent may include an anti-CD3 or anti-CD28 antibody at a concentration of from about 20 ng/mL-100 ng/mL. In certain embodiments, the concentration of anti-CD3 or anti-CD28 antibody may be about 20 ng/ml, about 30 ng/ml, about 40 ng/mL, about 50 ng/mL, about 60 ng/ml, about 70 ng/mL, about 80 ng/ml, about 90 ng/mL, or about 100 ng/mL. In certain embodiments, the T cell stimulating agent may also include Interleukin 2 at a concentration from about 10 IU/ml to 1000 IU/ml.

In some embodiments, a 3-day process of the present disclosure may comprise the following steps: apheresis material collection, a first wash step conducted on day 0, a second wash step conducted on day 0, an enrichment step conducted on day 0, a third wash step conducted on day 0, an activation step conducted on day 0 up to day 1, a viral transduction step conducted on-day0 up to day 4, and a fourth wash and concentration step conducted on day 1 up to day 3 (the day of harvest). The apheresis material may be fresh apheresis material, cryopreserved apheresis material, or cryopreserved T cells.

In some embodiments, the 3-day process comprises an activation step on day 0, day 1, or day 2. In some embodiments, the 3-day process may comprise a viral vector transduction step on day0, day 1, day 2, or day 3.

In some embodiments, the 3-day process may optionally comprise one, two, three, four, five, six, seven, eight or more wash steps. Each wash step may comprise the same wash procedure or a different wash procedure. In some embodiments, one or more wash steps may occur prior to the enrichment step. In some embodiments, the first wash may occur after the enrichment step. In some embodiments, the first wash may occur after the enrichment step and before the activation step. In some embodiments, the first wash may occur after both the enrichment and activation steps. In some embodiments, a wash step may be conducted on the day of harvest.

### Prepared Engineered Lymphocytes

The lymphocytes prepared, as demonstrated in the accompanying experimental examples, included higher ratios of lymphocytes expressing CCR7 and CD45RA.

In some embodiments, at least 80% of the transduced lymphocytes express CCR7 and CD45RA.

In some embodiments, at least 90% of the transduced lymphocytes express CCR7 and CD45RA.

Each type of T cells can be characterized with cell surface markers, as well known in the art. For instance, naïve T cells can be characterized as CCR7+, CD45RO−, and CD95—. Additional markers for naïve T cell include CD45RA+, CD62L+, CD27+, CD28+, CD127+, CD132+, CD25−, CD44−, and HLA-DR−.

Surface markers to stem memory T cells (Tscm) include, without limitation, CD45RO−, CCR7+, CD45RA+, CD62L+ (L-selectin), CD27+, CD28+, IL-7Ra+, CD95+, IL-2RP+, CXCR3+, and LFA−.

Surface markers for effector memory T cells (Tem) include, without limitation, CCR7−, CD45RO+ and CD95+. Additional marker for effector memory T cells is IL-2RB+. For central memory T cells (Tcm), suitable markers include CD45RO+, CD95+, IL-2RB+, CCR7+ and CD62L+. For effector T cells (Teff), suitable markers include CD45RA+, CD95+, IL-2RB+, CCR7− and CD62L−, without limitation.

The term “juvenile cells” as referred throughout includes one or more of naïve T cells, stem memory T cells (Tscm), and central memory T cells (Tcm). These cells are characterized, in part, by expression of CCR7+.

The harvested lymphocytes preferably include a good proportion that is CD3+ T cells. In some embodiments, at least 25%, 35%, 45%, 50%, 55%, 60%, 65%, 70%, 75%, 80%, 85% or 90% of the harvested lymphocytes are CD3+ T cells.

The harvested lymphocytes preferably include a good proportion that has been transduced. In some embodiments, at least 5%, 6%, 7%, 8%, 9%, 10%, 11%, 12%, 13%, 14% 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 42%, 44%, 45%, 46%, 47%, 48%, 49%, or 50% of the harvested lymphocytes are transduced with the vector. In some embodiments, each transduced lymphocyte includes at least a copy of the vector (or the included coding sequence) integrated to the host genome. In some embodiments, each transduced lymphocyte includes at least 2, 3, 4, 5, 6, 7, 8, 9 or 10 copies of the vector integrated to the host genome.

In some embodiments, the vector includes a transgene that encodes a polypeptide. The polypeptide, without limitation, may be a CAR or TCR. In some embodiments, the CAR or TCR includes an antigen binding molecule. The antigen binding molecule, in some embodiments, has binding specificity to an antigenic moiety. In some embodiments, the antigenic moiety is a tumor antigen (e.g., a protein or other molecule that is produced by a cancer cell).

In some embodiments, the vector includes more than one transgene that encodes for more than one CAR or TCR molecule that include antigen binding molecules with specificity to different antigenic moieties. In some embodiments, the vector includes more than one transgene that encodes for more than one CAR or TCR molecule that include antigen binding molecules with specificity to two different tumor antigens.

In some aspects, the antigenic moiety is an antigen associated with a cancer or a cancer cell. Such antigens may include, but are not limited to, 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2,

PSMA, RAGE-1, ROR1, RUI, RU2 (AS), surface adhesion molecule, survivin and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), GPC3 (Glypican 3), EphA2, GPC2, CD133, LGR5, TROP2, CD146, CLDN3, CLDN4, CLDN6, CLDN9, CLDN18.2, CD70, TnMUC1, Alkaline phosphatase (placental type), MUC16, MUC17, MART1, Pmel17, Melanoma-associated chondroitin sulfate proteoglycan, as well as any derivate or variant of these antigens.

A CAR of the present disclosure can include, in addition to the antigen-binding molecule, a hinge, a transmembrane domain, and/or an intracellular domain. In some embodiments, the intracellular domain can include a costimulatory domain and an activation domain.

A hinge may be an extracellular domain of an antigen binding system positioned between the binding motif and the transmembrane domain. A hinge may also be referred to as an extracellular domain or as a “spacer.” A hinge may contribute to receptor expression, activity, and/or stability. A hinge may also provide flexibility to access the targeted antigen. In some embodiments, a hinge domain is positioned between a binding motif and a transmembrane domain.

In some embodiments, the hinge is, is from, or is derived from (e.g., comprises all or a fragment of) an immunoglobulin-like hinge domain. In some embodiments, a hinge domain is from or derived from an immunoglobulin. In some embodiments, a hinge domain is selected from the hinge of IgG1, IgG2, IgG3, IgG4, IgA, IgD, IgE, or IgM, or a fragment thereof.

In some embodiments, the hinge is, is from, or is derived from (e.g., comprises all or a fragment of) CD2, CD3 delta, CD3 epsilon, CD3 gamma, CD4, CD7, CD8.alpha., CD8.beta., CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAMI), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAMI), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, or which is a fragment or combination thereof.

In some embodiments, the hinge is, is from, or is derived from (e.g., comprises all or a fragment of) a hinge of CD8 alpha. In some embodiments, the hinge is, is from, or is derived from a hinge of CD28. In some embodiments, the hinge is, is from, or is derived from a fragment of a hinge of CD8 alpha or a fragment of a hinge of CD28, wherein the fragment is anything less than the whole. In some embodiments, a fragment of a CD8 alpha hinge or a fragment of a CD28 hinge comprises an amino acid sequence that excludes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids at the N-terminus or C-Terminus, or both, of a CD8 alpha hinge, or of a CD28 hinge.

A “transmembrane domain” refers to a domain having an attribute of being present in the membrane when present in a molecule at a cell surface or cell membrane (e.g., spanning a portion or all of a cellular membrane). It is not required that every amino acid in a transmembrane domain be present in the membrane. For example, in some embodiments, a transmembrane domain is characterized in that a designated stretch or portion of a protein is substantially located in the membrane. Amino acid or nucleic acid sequences may be analyzed using a variety of algorithms to predict protein subcellular localization (e.g., transmembrane localization). The programs psort (PSORT.org) and Prosite (prosite.expasy.org) are exemplary of such programs.

A transmembrane domain may be derived either from any membrane-bound or transmembrane protein, such as an alpha, beta or zeta chain of a T-cell receptor, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CD5, CEACAMI, CRT AM, cytokine receptor, DAP-10, DNAMI (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

The intracellular domain (or cytoplasmic domain) comprises one or more signaling domains that, upon binding of target antigen to the binding motif, cause and/or mediate an intracellular signal, e.g., that activates one or more immune cell effector functions (e.g., native immune cell effector functions). In some embodiments, signaling domains of an intracellular domain mediate activation at least one of the normal effector functions of the immune cell. Effector function of a T cell, for example, may be cytolytic activity or helper activity comprising the secretion of cytokines. In some embodiments, signaling domains of an intracellular domain mediate T cell activation, proliferation, survival, and/or other T cell function. An intracellular domain may comprise a signaling domain that is an activating domain. An intracellular domain may comprise a signaling domain that is a costimulatory signaling domain.

Intracellular signaling domains that may transduce a signal upon binding of an antigen to an immune cell are known. For example, cytoplasmic sequences of a T cell receptor (TCR) are known to initiate signal transduction following TCR binding to an antigen (see, e.g., Brownlie et al., Nature Rev. Immunol. 13:257-269 (2013)).

In certain embodiments, suitable signaling domains include, without limitation, those of 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a,

CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CD5, CEACAMI, CRT AM, cytokine receptor, DAP-10, DNAMI (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-la/CD18), MHC class 1 molecule, NKG2C, NKG2D, NKp30, NKp44, NKp46, NKp80 (KLRF1), OX-40, PAG/Cbp, programmed death-1 (PD-1), PSGLI, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

A CAR can also include a costimulatory signaling domain, e.g., to increase signaling potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016). Signals generated through a TCR alone may be insufficient for full activation of a T cell and a secondary or co-stimulatory signal may increase activation. Thus, in some embodiments, a signaling domain further comprises one or more additional signaling domains (e.g., costimulatory signaling domains) that activate one or more immune cell effector functions (e.g., a native immune cell effector function described herein). In some embodiments, a portion of such costimulatory signaling domains may be used, as long as the portion transduces the effector function signal. In some embodiments, a cytoplasmic domain described herein comprises one or more cytoplasmic sequences of a T cell co-receptor (or fragment thereof). Non-limiting examples of such T cell co-receptors comprise CD27, CD28, 4-1BB (CD137), OX40, CD30, CD40, PD-1, ICOS, lymphocyte function-associated antigen-1 (LFA-1), MYD88, CD2, CD7, LIGHT, NKG2C, B7-H3, and a ligand that binds with CD83. An exemplary costimulatory protein has the amino acid sequence of a costimulatory protein found naturally on T cells, the complete native amino acid sequence of which costimulatory protein is described in NCBI Reference Sequence: NP 0.1. In certain instances, a CAR includes a 4-1BB costimulatory domain. In certain instances, a CAR includes a CD28 costimulatory domain. In certain instances, a CAR includes a DAP-10 costimulatory domain.

In some embodiments, the CAR further includes an ITAM. Examples of ITAM containing primary cytoplasmic signaling sequences that are of particular use in the disclosure include those derived from TCRzeta, FcRgamma, FcRbeta, CD3gamma, CD3delta, CD3epsilon, CD5, CD22 CD79a, CD79b and CD66d. In some embodiments, the ITAM includes CD3 zeta.

In some embodiments, the CAR molecule may be any anti-CD19 CAR molecule. In one aspect the anti-CD19 CAR includes an extracellular scFv domain, an intracellular and/or transmembrane, portion of a CD28 molecule, an optional extracellular portion of the CD2 molecule, and an intracellular CD3zeta domain as described in WO2015120096 or WO2016191755, each of which is incorporated herein in its entirety.

In certain embodiments, the anti-CD19 CAR may also include additional domains, such as a CD8 extracellular and/or transmembrane region, an extracellular immunoglobulin Fc domain (e.g., lgG1, lgG2, lgG3, lgG4), or one or more additional signaling domains, such as 41 BB, OX40 CD2 CD16, CD27, CD30 CD40, PD-1, ICOS, LFA-1, IL-2 Receptor, Fc gamma receptor, or any other costimulatory domains with immunoreceptor tyrosine-based activation motifs.

In certain embodiments, the cell surface receptor is an anti-CD19 CAR, such as FMC63-28Z CAR or FMC63-CD828BBZ CAR as set forth in Kochenderfer et al., J Immunother. 200 September; 32 (7): 689-702, “Construction and Preclinical Evaluation of an Anti-CD19 Chimeric Antigen Receptor,” the subject matter of which is hereby incorporated by reference for the purpose of providing the methods of constructing the vectors used to produce T cells expressing the FMC63-28Z CAR or FMC63-CD828BBZ CAR.

In some embodiments, the T cell that includes a CAR molecule is Yescarta® (axicabtagene ciloleucel). In some embodiments, the T cell that includes a CAR molecule is Tecartus® (brexucabtagene autoleucel).

In some embodiments, the engineered lymphocytes comprise a dual-targeted antigen binding system. Dual-targeted antigen binding systems may comprise bispecific CARs or TCRs and/or bicistronic CARs or TCRs. Bispecific and bicistronic CARs can comprise two binding motifs (in a single CAR molecule or in two CAR molecules, respectively). In some embodiments, the vector of the present disclosure encodes bicistronic and/or bispecific CARs (e.g., bicistronic and/or bispecific CARs that bind CD20 and CD19). In some embodiments, the bispecific CAR is one which targets CD19 and CD20 as described in WO2020123691, which is incorporated herein in its entirety.

In some embodiments, a pharmaceutical composition is provided that includes a population of engineered lymphocytes produced by the methods described herein. In certain embodiments, the pharmaceutical composition may also include a pharmaceutically acceptable carrier. A pharmaceutically acceptable carrier may be a pharmaceutically acceptable material, composition, or vehicle that is involved in carrying or transporting cells of interest from one tissue, organ, or portion of the body to another tissue, organ, or portion of the body. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

Treatments and Uses, and Optional Storage

The lymphocytes prepared by the instant methods, or the lymphocyte populations as disclosed herein, can be used for treating various diseases and conditions.

In some embodiments, if the lymphocytes are not immediately used, they can cryopreserved so that they can be used at a later date. Such a method may include a step of washing and concentrating the population of engineered lymphocytes with a diluent solution. In some aspects the diluent solution is normal saline, 0.9% saline, PlasmaLyte A (PL), 5% dextrose/0.45% NaCl saline solution (D5), human serum albumin (HSA), or a combination thereof. In some aspects, HSA may be added to the washed and concentrated cells for improved cell viability and cell recovery after thawing. In another aspect, the washing solution is normal saline and washed and concentrated cells are supplemented with HSA (5%). The method may also include a step of generating a cryopreservation mixture, wherein the cryopreservation mixture includes the diluted population of cells in the diluent solution and a suitable cryopreservative solution. In some aspects, the cryopreservative solution may be any suitable cryopreservative solution including, but not limited to, CryoStor®10 (BioLife Solution), mixed with the diluent solution of engineered lymphocytes at a ratio of 1:1 or 2:1.

In certain embodiments, HSA may be added to provide a final concentration of about 1.0-10% HSA in the cryopreserved mixture. In certain embodiments, HSA may be added to provide a final concentration of about 1.0%, about 2.0%, about 3.0%, about 4.0%, about 5.0%, about 6.0%, about 7.0%, about 8.0%, about 9.0%, or about 10.0% HSA in the cryopreserved mixture. In certain embodiments, HSA may be added to provide a final concentration of about 1-3% HSA, about 1-4% HSA, about 1-5% HSA, about 1-7% HSA, about 2-4% HSA, about 2-5% HSA, about 2-6% HSA, or about 2-7% HSA in the cryopreserved mixture. In certain embodiments, HSA may be added to provide a final concentration of about 2.5% HSA in the cryopreserved mixture. For example, in certain embodiments, cryopreservation of a population of engineered T cells may comprise washing cells with 0.9% normal saline, adding HSA at a final concentration of 5% to the washed cells, and diluting the cells 1:1 with CryoStor® CS10 (for a final concentration of 2.5% HSA in the final cryopreservation mixture). In some embodiments, the method also includes a step of freezing the cryopreservation mixture. In one aspect, the cryopreservation mixture is frozen in a controlled rate freezer using a defined freeze cycle at a cell concentration of between about 1e6 to about 1.5e7 cells per mL of cryopreservation mixture. The method may also include a step of storing the cryopreservation mixture in vapor phase liquid nitrogen.

Methods and uses are also provided, for treating a disease or pathological condition in a subject having the disease or pathological condition. In some embodiments, the method entails administering a therapeutically effective amount or therapeutically effective dose of the engineered lymphocytes to the subject. Pathogenic conditions that may be treated with engineered T cells that are produced by the methods described herein include, but are not limited to, cancer, viral infection, acute or chronic inflammation, autoimmune disease or any other immune-dysfunction.

As referred to herein, a “cancer” may be any cancer that is associated with a surface antigen or cancer marker, including, but not limited to, acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, central nervous system, B-cell leukemia, lymphoma or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, embryonal tumors, central nervous system, endometrial cancer, ependymoblastoma, ependymoma, esophageal cancer, esthesioneuroblastoma, ewing sarcoma family of tumors extracranial germ cell tumor, extragonadal germ cell tumor extrahepatic bile duct cancer, eye cancer fibrous histiocytoma of bone, malignant, and osteosarcoma, gallbladder cancer, gastric (stomach) cancer, gastrointestinal carcinoid tumor, small bowel and appendiceal cancer, colorectal cancer, anal cancer, gastrointestinal stromal tumors (GIST), soft tissue sarcoma, germ cell tumor, gestational trophoblastic tumor, glioma, meningioma, hairy cell leukemia, head and neck cancer, heart cancer, hepatocellular (liver) cancer, histiocytosis, hodgkin lymphoma, hypopharyngeal cancer, intraocular melanoma, islet cell tumors (endocrine pancreas), kaposi sarcoma, kidney cancer, langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone and osteosarcoma, medulloblastoma, medulloepithelioma, melanoma, merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CML), Myeloid leukemia, acute (AML), myeloma, multiple, myeloproliferative disorders, nasal cavity and paranasal sinus cancer, nasopharyngeal cancer, neuroblastoma, non-hodgkin lymphoma, small cell lung cancer, non-small cell lung cancer, oral cancer, oral cavity cancer, oropharyngeal cancer, osteosarcoma and malignant fibrous histiocytoma of bone, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, pregnancy and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, sézary syndrome, small cell lung cancer, small intestine cancer, soft tissue sarcoma, squamous cell carcinoma, squamous neck cancer, stomach (gastric) cancer, supratentorial primitive neuroectodermal tumors, t-cell lymphoma, cutaneous, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenström macroglobulinemia, Wilms Tumor, Nerave Sheath tumors, soft tissue and bone sarcomas, pediatric cancers, etc.

In some aspects, the cancer is a B cell malignancy. Examples of B cell malignancies include, but are not limited to, Non-Hodgkin's Lymphomas (NHL), Diffuse Large B Cell Lymphoma (DLBCL), Small lymphocytic lymphoma (SLL/CLL), Mantle cell lymphoma (MCL), Follicular lymphoma (FL), Marginal zone lymphoma (MZL), Extranodal (MALT lymphoma), Nodal (Monocytoid B-cell lymphoma), Splenic, Diffuse large cell lymphoma, B cell chronic lymphocytic leukemia/lymphoma, Burkitt's lymphoma and Lymphoblastic lymphoma. As referred to herein, a “viral infection” may be an infection caused by any virus which causes a disease or pathological condition in the host. Examples of viral infections that may be treated with the engineered T cells that are produced by the methods described herein include, but are not limited to, a viral infection caused by an Epstein-Barr virus (EBV); a viral infection caused by a hepatitis A virus, a hepatitis B virus or a hepatitis C virus; a viral infection caused by a herpes simplex type 1 virus, a herpes simplex type 2 virus, or a herpes simplex type 8 virus, a viral infection caused by a cytomegalovirus (CMV), a viral infection caused by a human immunodeficiency virus (HIV), a viral infection caused by an influenza virus, a viral infection caused by a measles or mumps virus, a viral infection caused by a human papillomavirus (HPV), a viral infection caused by a parainfluenza virus, a viral infection caused by a rubella virus, a viral infection caused by a respiratory syncytial virus (RSV), or a viral infection caused by a varicella-zostser virus. In some aspects, a viral infection may lead to or result in the development of cancer in a subject with the viral infection (e.g., HPV infection may cause or be associated with the development of several cancers, including cervical, vulvar, vaginal, penile, anal, oropharyngeal cancers, and HIV infection may cause the development of Kaposi's sarcoma). Examples of chronic inflammation diseases, autoimmune diseases or any other immune-dysfunctions that may be treated with the engineered T cells produced by the methods described herein include, but are not limited to, multiple sclerosis, lupus, and psoriasis.

Additional examples of chronic inflammation diseases, autoimmune diseases or any other immune-dysfunctions that may be treated with the engineered T cells produced by the methods described herein include rheumatoid arthritis, allergies, asthma, Crohn's disease, IBD, IBS, fibromyalga, mastocytosis, and Celiac disease.

The term “treat,” “treating” or “treatment” as used herein with regard to a condition or disease may refer to preventing a condition or disease, slowing the onset or rate of development of the condition or disease, reducing the risk of developing the condition or disease, preventing or delaying the development of symptoms associated with the condition or disease, reducing or ending symptoms associated with the condition or disease, generating a complete or partial regression of the condition or disease, or some combination thereof.

A “therapeutically effective amount” or a “therapeutically effective dose” is an amount of engineered lymphocytes that produce a desired therapeutic effect in a subject, such as preventing or treating a target condition or alleviating symptoms associated with the condition by killing target cells. The most effective results in terms of efficacy of treatment in a given subject will vary depending upon a variety of factors, including but not limited to the characteristics of the engineered lymphocytes (including longevity, activity, pharmacokinetics, pharmacodynamics, and bioavailability), the physiological condition of the subject (including age, sex, disease type and stage, general physical condition, responsiveness to a given dosage, and type of medication), the nature of any pharmaceutically acceptable carrier or carriers in any composition used, and the route of administration. A therapeutically effective dose of engineered lymphocytes also depends on the cell surface receptor that is expressed by the lymphocytes (e.g., the affinity and density of the cell surface receptors expressed on the cell), the type of target cell, the nature of the disease or pathological condition being treated, or a combination of both.

In some aspects, a therapeutically effective dose of engineered lymphocytes is fewer than about 2 million engineered lymphocytes per kilogram of body weight of the subject in need of treatment (cells/kg). Therefore, in some aspects, a therapeutically effective dose of engineered lymphocytes is from about 10,000 to about 2,500,000 engineered lymphocytes/kg. In certain embodiments, a therapeutically effective dose of engineered lymphocytes is from about 10,000 to about 1,500,000 engineered lymphocytes/kg. In certain embodiments, the therapeutically effective dose is about 20,000 to about 1,200,000 engineered lymphocytes/kg. In certain embodiments, the therapeutically effective dose is about 20,000 to about 1,000,000 engineered lymphocytes/kg. In certain embodiments, the therapeutically effective dose is about 20,000 to about 500,000 engineered lymphocytes/kg. In certain embodiments, the therapeutically effective dose is about 20,000 to about 400,000 engineered lymphocytes/kg. In certain embodiments, the therapeutically effective dose is about 40,000 to about 400,000 engineered lymphocytes/kg. In certain embodiments, the therapeutically effective dose is about 50,000 to about 200,000engineered lymphocytes/kg. In certain embodiments, the therapeutically effective dose is about 50,000 to about 100,000 engineered lymphocytes/kg.

In some aspects, a therapeutically effective dose of engineered lymphocytes is from about 1,600,000 to about 2,500,000 engineered lymphocytes per kilogram of body weight of the subject in need of treatment (cells/kg). In some embodiments, the therapeutically effective dose of engineered lymphocytes is from about 2,000,000 to about 2,400,000 engineered lymphocytes per kilogram of body weight of the subject in need of treatment (cells/kg).

In some embodiments, the T cells administered are Yescarta® (axicabtagene ciloleucel). In some embodiments, the T cells administered are Tecartus® (brexucabtagene autoleucel).

The following examples are intended to illustrate various embodiments of the invention. As such, the specific embodiments discussed are not to be construed as limitations on the scope of the invention. For example, although the Examples below are directed to T cells transduced with an anti-CD19 chimeric antigen receptor (CAR), one skilled in the art would understand that the methods described herein may apply to T cells transduced with any CAR. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1

### An Automated, One-Step Process Depleting CD45RO Positive T Cells Enables the Production of CAR-T Cell Product With High Purity of T Memory Stem Cells

### Leukapheresis Processing

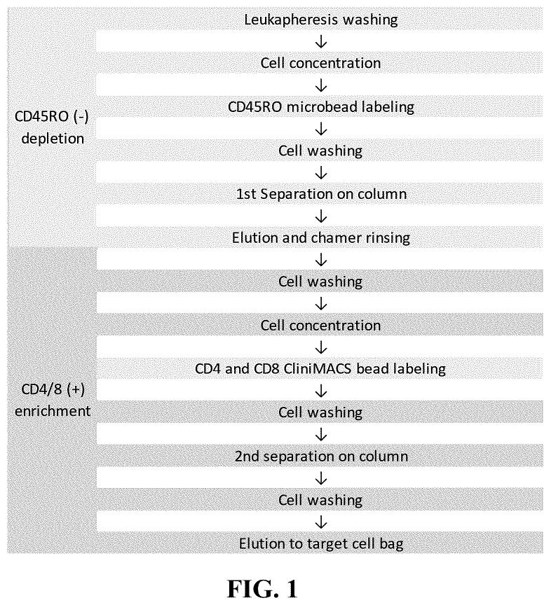

On day 0, fresh leukapheresis are processed to enrich for CD4+, CD8+, and CD45RO− subpopulations of T cells using CliniMACS® Prodigy and T310 kit (or T520 kit) as illustrated in the FIG. 1. Specifically, the leukapheresis bag containing 6-10 e9 white blood cells and red blood cells is sterile welded onto the T310 kit or T520 kit's application bag, followed by washing with CliniMACS® PBS/EDTA buffer supplemented with human serum albumin at 0.5%, and blocking with 5% IgG solution. The cells are then labeled with 12-20 ml CD45RO microbead (Miltenyi Biotech, Cat 130-046-001) for 30 mins at RT. The positive fraction of cells goes to non-target cell bag. The negative fraction of cells is collected, washed with CliniMACS® PBS/EDTA buffer+0.5% human serum albumin, and labeled with 15 ml CliniMACS® CD4 GMP MicroBeads (Miltenyi Biotech, Cat 170-076-702) and CliniMACS® CD8 GMP MicroBeads (Miltenyi Biotech, Cat 170-076-703) at 1:1 ratio for 30 mins at room temperature. The positive fraction of cells is then washed and eluted into target cell bag in saline + 0.5% human serum albumin. Isolated cells are then either formulated in CryoStor® CS5 (STEMCELL Technologies Cat 07953) at 50e6 cells/ml and cryopreserved in a rate-controlled manner at −1° C./min until −80° C. or activated immediately with anti-CD3 and anti-CD28 as described below.

### T Cell Activation

On day 0, a PermaLife™ cell culture bag (PL120) is incubated with 55 ml Hank's Balanced Salt Solution (HBSS) containing anti-CD3 at 1.24 μg/ml for 3 hrs at 37° C. The cell culture bag is rinsed with HBSS followed by inoculation of 1.72e8 total viable CD45RO(−) T cells at 138 ml of CTS Optmizer™ cell culture media supplemented with anti-CD28 (1.0μg/ml), CTS Immune Cell SR (2.5%), and GlutaMax™ (2 mM), and interlukin-2 (300 IU/ml). The cells are activated for a total of 72 hours in PL 120 at 5% CO₂ and 37° C. incubator before removal of activators.

### T Cell Transduction

On day 1, cells are counted and transduced with lentiviral vector based on the total viable cell counts and functional titer of the lentiviral vector.

### T Cell Expansion

Cells were maintained in PL120 cell culture bag in complete culture media in a 5% CO₂ and 37° C. incubator until being harvested on day3.

### Harvest, Formulation, and Cryopreservation

On day 3, cells and cell culture supernatant are harvested and washed using Sepax C-Pro Culture Wash Pro application using a CT 60.1 kit, through which, cells went through one around of volume reduction followed by another around of washing using saline supplemented with 0.5% human serum albumin. Cells were formulated in CryoStor® CS5 (STEMCELL Technologies Cat 07953) at 50e6 cells/ml. Finally, cells were cryopreserved in a rate-controlled manner at −1° C./min until −80° C.

### Example 2

### An Automated Process Banking CD45RO(−) Leukapheresis as an Alternative Starting Material for Manufacturing Genetic Engineered T Cells Enables T Cell Product Enriched With Tscm Subset

### Leukapheresis Processing

On day 0, fresh leukapheresis are processed to deplete CD45RO (+) population using CliniMACS® Prodigy and T310 kit. Specifically, the bag containing 6-10 e9 leukapheresis is sterile welded onto the T310 kit's application bag, followed by washing with CliniMACS® PBS/EDTA buffer supplemented with human serum albumin at 0.5%, and blocking with 5% IgG solution. The cells are then labeled with 12-20 ml CD45RO Antibody, anti-human, Biotin (Miltenyi Biotech, Cat 130-113-548) for 15-30 mins at RT, followed by labeling with 7.5 ml of CliniMACS® Anti-Biotin GMP microbeads (Milteniyi Biotech, Cat 170-076-709). The cells then go through column separation after labeling and the non-labeled cells (negative fraction) are washed, formulated in CryoStor® CS5 in one or multiple freezing bags at 50e6/ml, and cryopreserved in a rate-controlled manner. These CD45RO(−) leukapheresis are then banked as the start material for manufacturing genetic engineered T cell in a separate process.

On day 1, a bag of frozen CD45RO(−) leukapheresis at 3-5 e9 mononuclear TVC are thawed and sterile welded onto CliniMACS® Prodigy Tubing Set 520 (Miltenyi Biotech Cat 170-076-600) on a CliniMACS® Prodigy. The cells then go through TCT application minus activation and expansion unit operations. Specifically, cells are washed with CliniMACS® PBS/EDTA buffer with 0.5% human serum albumin and concentrated in the same reagent followed by labeling with 15 ml CliniMACS® CD4 GMP MicroBeads (Miltenyi Biotech, Cat 170-076-702) and CliniMACS® CD8 GMP MicroBeads (Miltenyi Biotech, Cat 170-076-703) at 1:1 ratio for 30 mins at RT. The positive fraction of cells is collected, washed with CliniMACS® buffer+0.5% human serum albumin, and eluted into target cell bag in saline+0.5% human serum albumin. Isolated CD45RO (−) CD4 (+) CD8 (+) cells immediately proceed with T cell activation unit operation as described below.

### T Cell Activation

On day 1, a PermaLife™ cell culture bag (PL120) is incubated with 55 ml Hank's Balanced Salt Solution (HBSS) containing anti-CD3 at 1.24 μg/ml for 3 hrs at 37° C. The cell culture bag is rinsed with HBSS followed by inoculation of 1.72E8 total viable CD45RO(−) T cells at 138 ml of CTS Optmizer™ cell culture media supplemented with anti-CD28 (1.0μg/ml), CTS Immune Cell SR (2.5%), and GlutaMax™ (2 mM), and interlukin-2 (300IU/ml). The cells are activated for a total of 72 hours in PL120 at 5% CO₂ and 37° C. incubator before removal of activators.

### T Cell Transduction

On day 2, cells are counted and transduced with lentiviral vector based on the total viable cell counts and functional titer of the lentiviral vector.

### T Cell Expansion

Cells were maintained in PL120 cell culture bag in complete culture media in a 5% CO₂ and 37° C. incubator until being harvested on day4.

### Harvest, Formulation, and Cryopreservation

On day 4, cells and cell culture supernatant are harvested and washed using Sepax C-Pro Culture Wash Pro application using a CT 60.1 kit, through which, cells went through one around of volume reduction followed by another around of washing using saline supplemented with 0.5% human serum albumin. Cells were formulated in CryoStor® CS5 (STEMCELL Technologies Cat 07953) at 50e6 cells/ml. Finally, cells were cryopreserved in a rate-controlled manner at −1° C./min until −80° C.

### Example 3

### Results of Manufacturing CAR T-Cells Using the Processes of Example 1

The following example provides results from use of the methods described in Example 1.

The process for enriching starting T cell material for the manufacturing of CAR T cell is described in FIG. 1. Specially, fresh leukapheresis was washed and labeled with anti-hCD45RO microbeads and separated through the 1^{st} magnetic column. The negative fraction was then labeled with anti-hCD4 and anti-hCD8 microbeads and separated through the 2^{nd} magnetic column. The positive fraction from 2^{nd} magnetic column (CD4+CD8+CD45RO-T cells) were used as one the arms in the CAR T cell manufacturing process and harvested and cryopreserved 72 hours following T cell activation.

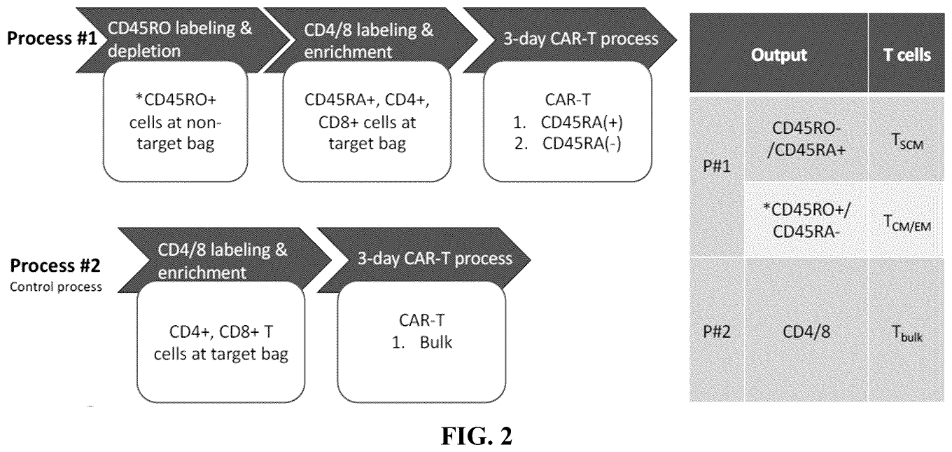

For comparison, two additional arms were included in the study as illustrated in FIG. 2. Process #1 depletes CD45RO+ cells prior to enriching CD4+ and CD8+ T cells, followed by T cell activation on day0, lentivirus transduction on day1, and harvest on day3. Process #2 is the conventional process that enriches CD4+ and CD8+ T cells without CD45RO+ depletion, followed by T cell activation on day0, lentivirus transduction on dayl, and harvest on day3. Process #1 (P #1) leads to two arms which are arm Tscm (product) and arm Tcm/em (non-product to serve as a control) and Process #2 (P #2) leads to a competing arm Tbulk.

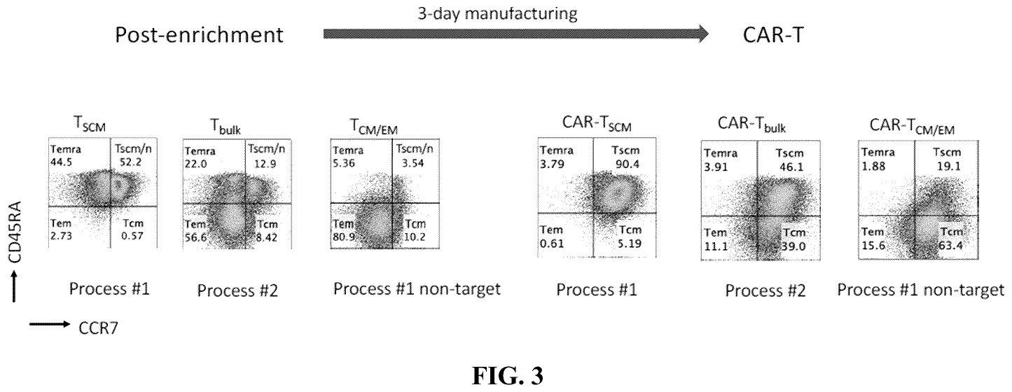

The T memory stem cell phenotype was investigated based on the expressions of CD45RA and CCR7. Additional gating strategies such as CD3 and CD95 were used but not shown. As shown in FIG. 3, the CD45RA-population was depleted post-leukapheresis processing (left) in the Tscm arm and enriched in the Tcm/em arm. The Tscm arm resulted in 90% Tscm by harvest whereas the Tcm/em and Tbulk arms had Tscm population at 19% and 46% by harvest, respectively.

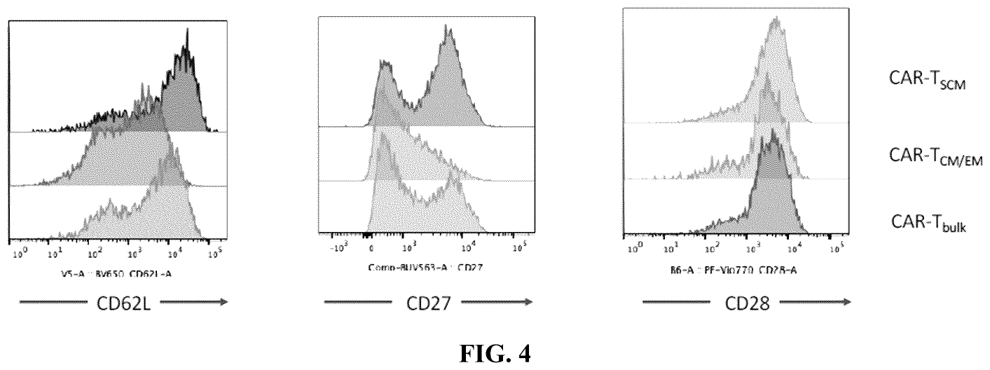

Additional memory markers were investigated as a confirmation of the juvenile phenotype of the Tscm cells, including CD62L, CD27, and CD28. As shown in FIG. 4, CAR-T cell from the Tscm arm has higher expression of CD62L and CD27, and comparable expression of CD28 compared to CAR-T cells from the Tem/em and Tbulk arms, suggesting a superior memory phenotype.

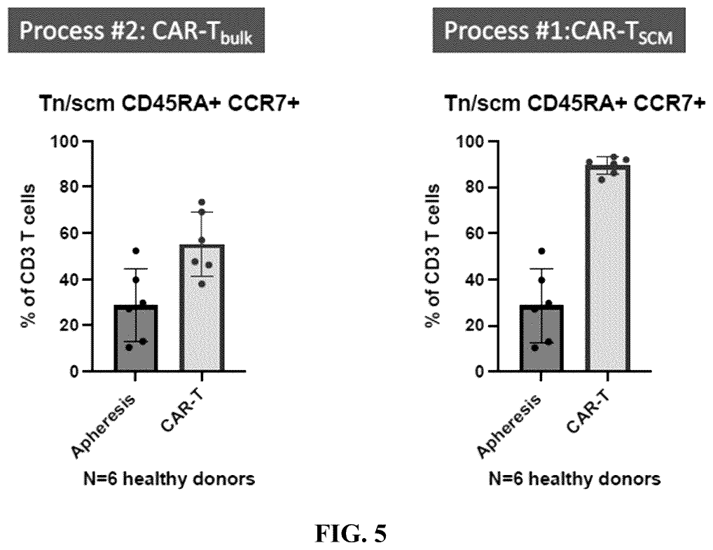

To confirm the robustness and reproducibility of the process, % Tscm from 6 donor leukapheresis and CAR T product were plotted together from the Process #1 Tscm arm (right) and the Process #2 Tbulk arm (left) in FIG. 5. In the Tbulk arm, naïve T cell variation (10% to 50%) from leukopahersis led to inconsistent % Tscm of CAR T final product manufactured (40% to 70%). However, independent of the starting % Tnaive cells from leukapheresis, the Tscm arm from Process #1 consistently led to ˜90% Tscm in the final CAR T product as a result of CD45RO depletion step implemented prior to T cell activation.

T cells were transduced with lentivirus encoding a CD19/20 bicistronic CAR on day 1 and CAR expression was characterized on day3 post-harvest. Details of the CD19/20 bicistronic CAR are provided in patent publication WO2020123691, which is hereby incorporated in its entirety.

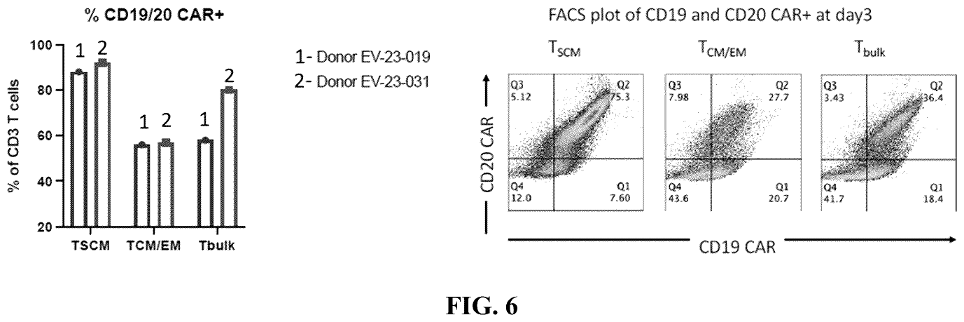

Possibly as a result of better T cell activation and/or more accessible locus for genetic editing of T cell, transduction efficiency as quantified by overall expression of the anti-CD19 and anti-CD20 CAR was significantly higher in the Tscm arm compared with other two arms (FIG. 6 left), particularly higher in the population that is double positive for both of the anti-CD19 and the anti-CD20 binding domains of the CAR (FIG. 6 right).

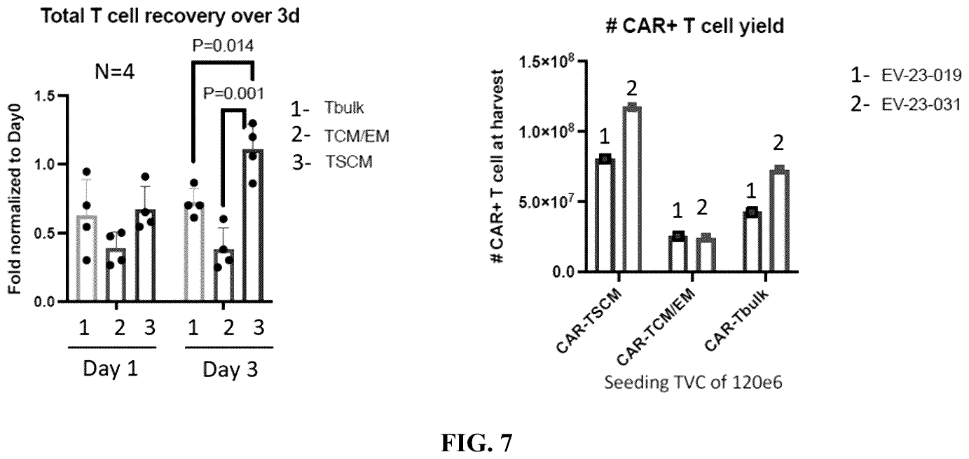

As a result of better T cell activation, yield of total T cell at harvest (Day3) is consistently better in the Tscm arm compared to other two arms (n=4), of which the Tem/em arms had the lowest T cell yield (FIG. 7 left). Combined with higher percentage of CAR+ T cells and better total T cell yield in the Tscm arm, the CAR+ T cell yield is closed to 2-fold higher compared to the Tcm/em arm (FIG. 7 right).

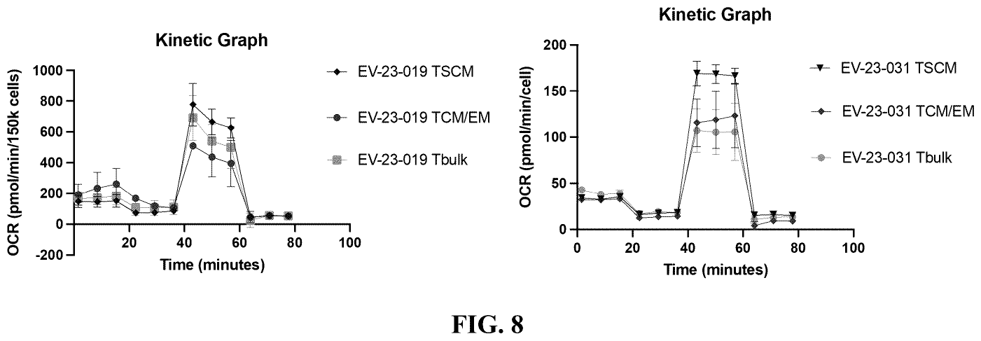

T cell products from 2 donors were further characterized by Seahorse T cell metabolic profiling assay (FIG. 8). T cells from Tscm arm has the greatest spare respiration capacity indicated by highest maximal oxygen consumption upon uncoupling of mitochondria, suggesting a better metabolic fitness of the T cell product from Tscm arm.

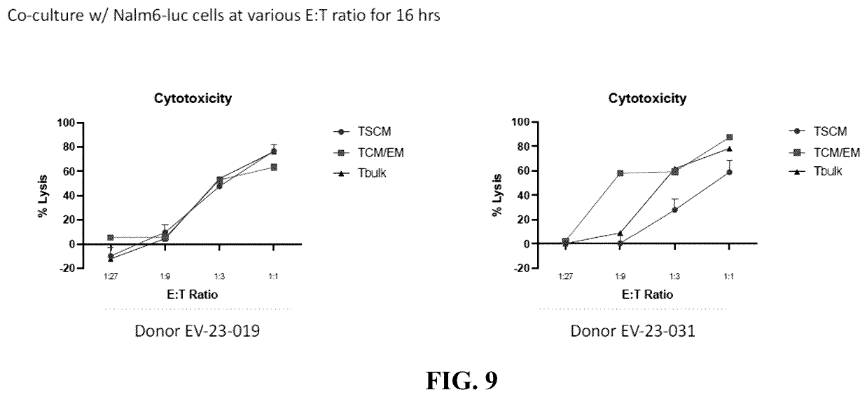

CAR T cells were cocultured with Nalm6 target cells at different effector to target cell ratio from 1:27 to 1:1. Cytotoxicity was quantified by the % of killing of Nalm6 cells overnight. It is shown in FIG. 9, CAR T cells from the Tscm arm have comparable cytotoxicity from the two donors tested when compared to CAR T cells from the Tbulk arm.

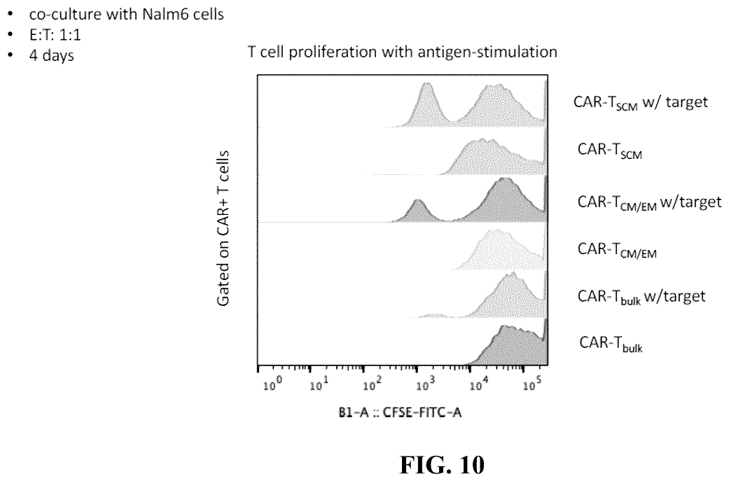

In a similar coculture assay where CAR T cells are cocultured with Nalm6 target cells at effector to target ratio at 1:1 following DNA labeling with CFSE, it is shown in FIG. 10 that CAR T cells from the Tscm arm proliferated significantly better than CAR T cells from the other two arms over 4 days of antigen stimulation.

### Example 4

### Characterization of CAR T Cell Product from Additional Donors Using the Process of Example 1

The following example provides additional results using methods described in Example 1. Additional 5 healthy donor-derived apheresis were processed as per the methods described in Example 1. CAR T cells were generated by transducing with lentivirus encoding an a CD19/20 CAR derived either from a process which first depletes CD45RO+ cells followed by enriching for CD4+ and CD8+ cells (CD45RO depletion process) or from a process which enriches for CD4+ and CD8+ T cells (CD4,8 enrichment process). Alternatively, CAR T cells were generated by enriching CD45RO+ cells from apheresis as a control. CAR T cells were harvested, formulated, and cryopreserved on day3. In some studies, CAR T cells from a CD4,8 enrichment process are harvested on day6 or day8 to serve as controls.

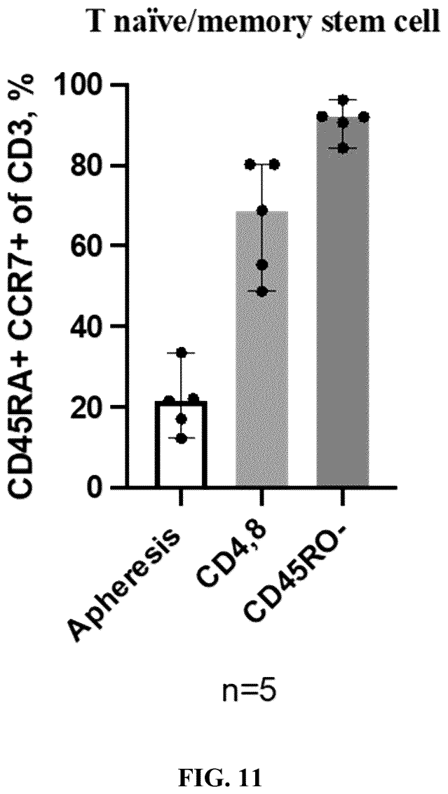

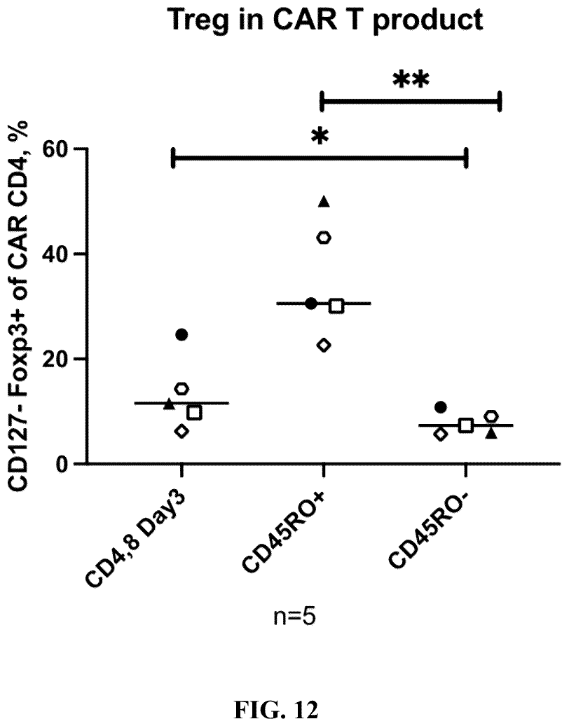

The percentage of T naïve cells that are characterized as CD45RA+ and CCR7+ from incoming healthy donor-derived apheresis ranges from 10% to 35% (FIG. 11). As a result of this donor-to-donor variations, the percentage of TscM in CAR T cell product from CD4,8 enrichment process across 5 donors is from 50% to 80%. In comparison, the percentage of TsCM in CAR T cell product from the CD45RO depletion process is consistently around 90%. In addition to having a higher percentage of TsCM, the percentage of Treg cells in the CAR T product are consistently lower when CAR T cells are generated from a CD45RO depletion process compared to a CD4,8 and CD45RO enrichment process (FIG. 12).

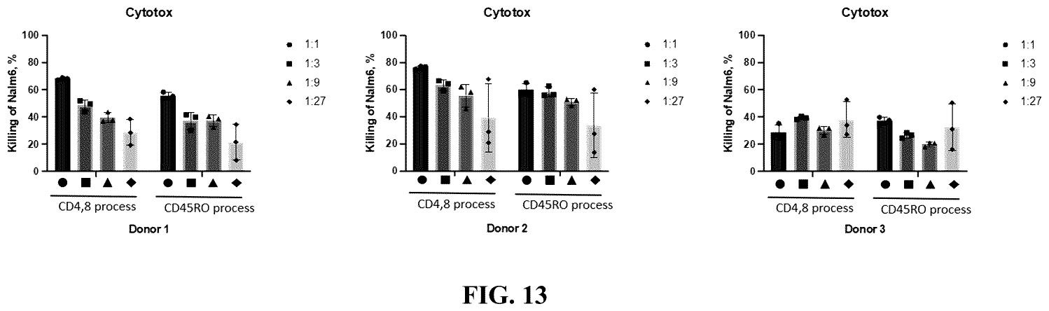

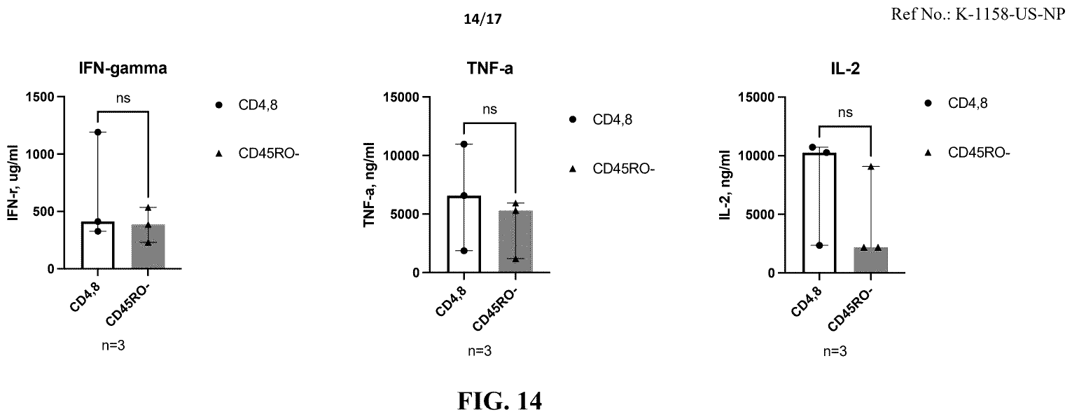

CAR T cells from 3 healthy donors were cocultured with Nalm6-luc target cells at different effector to target cells (E:T) ratios, i.e., 1:1, 1:3, 1:9, 1:27, for 24 hours. The percentage of killing of target cells were measured based on luciferase intensity. Cytokines from coculture supernatant, including IFN-gamma, TNF-alpha, and IL-2, were measured. Cytotoxicity and cytokine level are similar between CAR T cells generated from CD4,8 enrichment process and CAR T cells generated from CD45RO depletion process (FIG. 13 & FIG. 14).

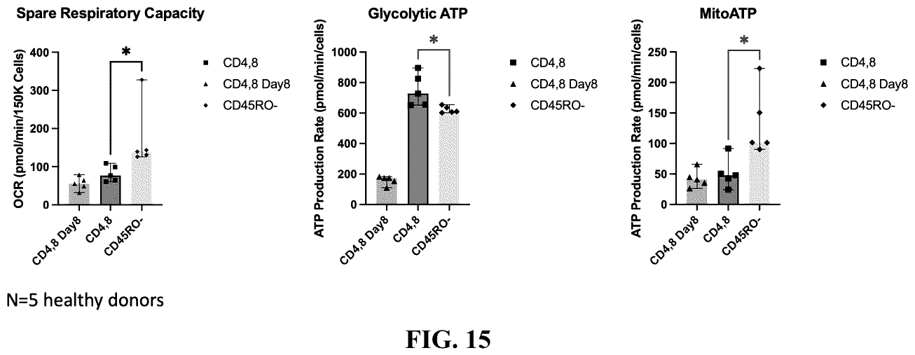

Metabolic fitness of CAR T cells from the CD4,8 enrichment process and the CD45RO depletion process are compared in a Seahorse T cell metabolic profiling assay where viable T cells are sequentially dosed with oligomycin, BAM15, and rotenone/antimycin A and oxygen consumption rate (OCR) and ATP product rate are measured. CAR T cells enriched with TsCM from the CD45RO depletion process showed a higher respiration capacity, lower glycolytic ATP, and more mitochondrial ATP, indicating an improved metabolic fitness compared to CAR T cells from CD4,8 enrichment process (FIG. 15).

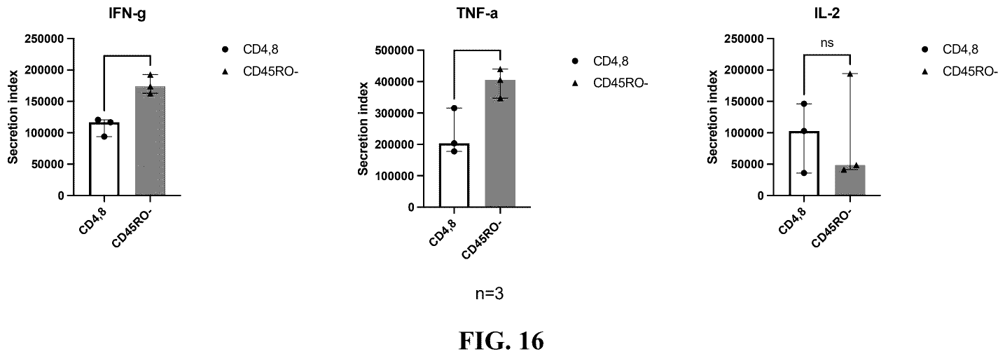

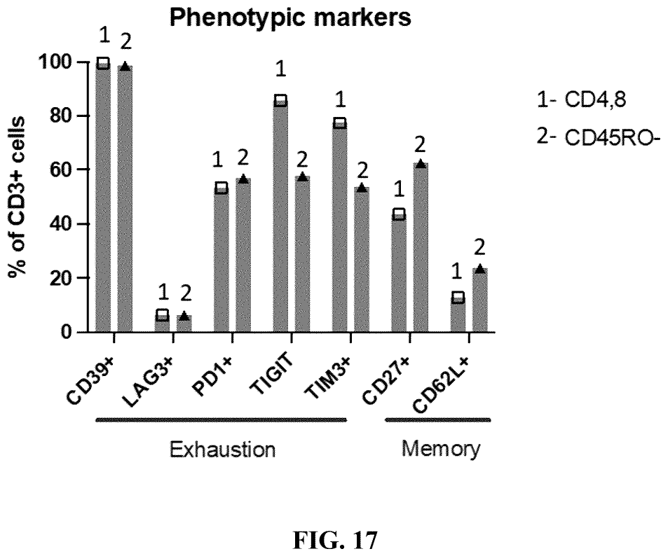

CAR T cells were also studied in a repeated antigen stimulation assay where CAR T cells were co-cultured with Nalm6 cells for every 3-4 days when new Nalm6 cells were added to the co-culture for 5 times over a period of 2.5 weeks. At the end of the assay, CAR T cells who are enriched with TscM from CD45RO depletion process showed higher cytokine secretion capacity of IFN-gamma, TNF-alpha, and IL-2, than CAR T cells from CD4,8 enrichment process (FIG. 16). Consistently, expression level of memory markers, including CD27 and CD62L, and exhaustion markers, including TIGIT and TIM3 are lower in CAR T cells from CD45RO depletion process compared to CAR T cells from CD4,8 enrichment process (FIG. 17).

While a number of embodiments have been described, it is apparent that the disclosure and examples may provide other embodiments that utilize or are encompassed by the compositions and methods described herein. Therefore, it will be appreciated that the scope of is to be defined by that which may be understood from the disclosure and the appended claims rather than by the embodiments that have been represented by way of example.

1. A method for manufacturing transduced lymphocytes, comprising:
depleting a population of cells expressing CD45RO from a sample of lymphocytes obtained from a donor subject;
activating a population of lymphocytes expressing at least one of CD4 and CD8 from the sample of lymphocytes by stimulating the population of lymphocytes expressing at least one of CD4 and CD8 with at least one T cell stimulating agent; and
incubating the population of lymphocytes expressing at least one of CD4 and CD8 with a polynucleotide vector to transduce the population of lymphocytes expressing at least one of CD4 and CD8 lymphocytes to produce transduced lymphocytes.

2. The method of claim 1, wherein depleting the population of cells expressing CD45RO from the sample of lymphocytes comprises:
contacting the sample of lymphocytes with an anti-hCD45RO antibody and bead conjugate so as to generate a labeled population of cells expressing CD45RO; and
separating the labeled population of cells expressing CD45RO from the sample of lymphocytes.

3. The method of claim 1, further comprising enriching the sample of lymphocytes for a population of lymphocytes expressing at least one of CD4 and CD8 comprising:
contacting the sample of lymphocytes with at least one of an anti-CD4 antibody and bead conjugate, and an anti-CD8 antibody and bead conjugate so as to generate a labeled population of cells expressing at least one of CD4 and CD8; and
isolating the labeled population of cells expressing at least one of CD4 and CD8 from the sample of lymphocytes.

4. The method of claim 1, wherein the activating is done prior to incubating the population of lymphocytes expressing at least one of CD4 and CD8 with the polynucleotide vector, wherein the at least one T cell stimulating agent comprises an anti-CD3 antibody, an anti-CD28 antibody, or Interleukin-2, and wherein the activating is carried out in a closed system.

5. (canceled)

6. (canceled)

7. The method of claim 1, wherein the population of lymphocytes expressing at least one of CD4 and CD8 are incubated with the at least one T cell stimulating agent for up to 72 hours and wherein the incubating is carried out in a closed system.

8. (canceled)

9. The method of claim 1, wherein at least 80% of the transduced lymphocytes express CCR7 and CD45RA.

10. (canceled)

11. The method of claim 1, wherein at most 10% of the transduced lymphocytes are a combination of effector memory T cells (TEM) and central memory T cells (TEM).

12. (canceled)

13. The method of claim 1, further comprising:
culturing the sample comprising the transduced lymphocytes for up to 72 hours before the lymphocytes are harvested to produce a harvested sample.

14. (canceled)

15. (canceled)

16. The method of claim 13, wherein the culturing is carried out in a closed system, and wherein the closed system has an inner surface area of at least 170 cm².

17. (canceled)

18. The method of claim 16, wherein the closed system has an inner surface coated with a recombinant human fibronectin, wherein the coating is carried out with a solution that comprises about 1-10 μg/ml of the recombinant human fibronectin.

19. The method of claim 16, wherein the sample in the closed system comprises at least 1.2×10⁸ lymphocytes.

20. (canceled)

21. The method of claim 13, further comprising, following the harvesting, administering the harvested lymphocytes to a subject in need thereof or freezing the harvested lymphocytes.

22. The method of claim 21, wherein a total of 5,000 to 1,000,000 harvested lymphocytes per kilogram of the subject in need thereof are administered to the subject.

23. The method of claim 1, wherein the sample of lymphocytes derived from the donor subject are washed leukapheresis cells, peripheral blood mononuclear cells (PBMCs) or T cells.

24. The method of claim 1, wherein the polynucleotide vector is a viral vector, wherein the viral vector is a retroviral vector or a lentiviral vector, wherein the polynucleotide vector encodes a chimeric antigen receptor (CAR) or a T cell receptor (TCR), and wherein the CAR or the TCR recognizes a tumor antigen.

25. (canceled)

26. (canceled)

27. (canceled)

28. The method of claim 24, wherein the tumor antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, Fc receptor-like protein 5, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen;
CD38, insulin growth factor (IGF1)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, TACI, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), GPC3 (Glypican 3), EphA2, GPC2, CD133, LGR5, TROP2, CD146, CLDN3, CLDN4, CLDN6, CLDN9, CLDN18.2, CD70, TnMUC1, Alkaline phosphatase (placental type), MUC16, MUC17, MART1, Pmel17, Melanoma-associated chondroitin sulfate proteoglycan, as well as any derivate or variant of these antigens.

29. A population of cells prepared by the method of claim 1, wherein at least 80% of the population of cells express CCR7 and CD45RA, and wherein at most 10% of the population of cells are a combination of effector memory T cells (TEM) and central memory T cells (TEM).

30. A pharmaceutical composition comprising the population of cells of claim 29.

31. A method for administering T cells to a subject, comprising injecting to the subject a harvested sample prepared by the method of claim 1, and wherein the subject has a cancer.

32. (canceled)

33. The method of claim 31, wherein the cancer is a lung cancer, a GI cancer, a breast cancer, a gynecologic malignancy, a genitourinary malignancy, a neurologic tumor, a melanoma, a sarcoma, a pediatric cancer, an endocrine malignancy, Kaposi sarcoma, a Non-Hodgkin's Lymphoma, or mesothelioma.

34. (canceled)

35. (canceled)
